(19) United States
(12) Patent Application Publication
(10) Pub. No.: US 2019/0352361 A1
(43) Pub. Date: Nov. 21, 2019

(54) TISSUE-BASED BIOLOGICS FOR THE TREATMENT OF INFLAMMATORY AND AUTOIMMUNE DISORDERS

(71) Applicant: The Brigham and Women`s Hospital, Inc., Boston, MA (US)

(72) Inventor: Rachael Clark, Belmont, MA (US)

(21) Appl. No.: 16/469,995

(22) PCT Filed: Dec. 15, 2017
(86) PCT No.: PCT/US2017/066785

§ 371 Date: Jun. 14, 2019

Related U.S. Application Data
(60) Provisional application No. 62/434,593, filed on Dec. 15, 2016.
(60) Provisional application No. 62/476,340, filed on Mar. 24, 2017.

(51) Int. Cl. C07K 14/525; C07K 14/54; C07K 14/57; C07K 14/715; C07K 14/78; C07K 16/24; A61P 37/06

(52) U.S. Cl. CPC C07K 14/525; C07K 14/54; C07K 14/57; C07K 14/7156; C07K 14/7155; C07K 2317/76; C07K 14/78; C07K 16/241; A61P 37/06; C07K 2317/31; C07K 14/7151

(57) ABSTRACT

Described herein are tissue-targeted biologies comprising collagen VII binding domains linked to cytokine binding domains, and their use in suppression of inflammation.

This application claims the benefit of U.S. Patent Application Ser. Nos. 62/434,593, filed on Dec. 15, 2016, and 62/476,340, filed on Mar. 24, 2017. The entire contents of the foregoing are hereby incorporated by reference.

### FEDERALLY SPONSORED RESEARCH OR DEVELOPMENT

This invention was made with Government support under Grant No. AR063962 awarded by the National Institutes of Health. The Government has certain rights in the invention. CL CLAIM OF PRIORITY

### SEQUENCE LISTING

The instant application contains a Sequence Listing which has been submitted electronically in ASCII format and is hereby incorporated by reference in its entirety. Said ASCII copy, created on Nov. 30, 2017, is named 40175-0012WO1_SL.txt and is 109,877 bytes in size.

### TECHNICAL FIELD

Described herein are tissue targeted biologics comprising collagen VII-binding domains linked to cytokine-binding domains, and their use in suppression of inflammation.

### BACKGROUND

Biologic medications have revolutionized the treatment of a number of inflammatory and autoimmune disorders. For example, agents targeting TNFα (including etanercept, infliximab, adalimumab, certolizumab and golimumab) and IL-12/IL-23 (ustekinumab and secukinumab) have received regulatory approval for treating conditions including RA, Crohn's disease, ulcerative colitis, psoriatic arthritis, ankylosing spondylitis, chronic plaque psoriasis, moderate-to-severe chronic psoriasis, moderate-to-severe hidradenitis suppurativa, juvenile idiopathic arthritis, and noninfectious uveitis, many of which previously lacked any effective treatment. However, the use of antibody-based biologics is typically limited to patients with severe disease because these drugs significantly increase the risk of potentially fatal infections.

### SUMMARY

Described herein are collagen VII-targeted constructs and their use, e.g., in treating inflammation in tissues that express collagen VII, e.g., skin, lung, and gut.

In a first aspect, provided here are collagen-targeted constructs comprising: (i) a collagen binding domain that binds specifically to human collagen VII; and (ii) a cytokine binding domain that binds specifically to an inflammatory cytokine and inhibits binding of the cytokine to its receptor, wherein the collagen binding domain and the cytokine binding domain are bound to each other.

In some embodiments, (i) and (ii) are each capable of binding to its cognate antigen at the same time.

In some embodiments, the cytokine binding domain binds specifically to an inflammatory cytokine selected from the group consisting of tumor necrosis factor alpha (TNFα), Interleukin 17A (IL-17A), IL-12, IL-23, IL-6, IL-4, and Interferon gamma (IFNγ).

In some embodiments, one or both of the collagen binding domain and the cytokine binding domain are antibodies or antigen-binding fragments thereof. In some embodiments, the antibody or antigen-binding fragment thereof is an Fv fragment, a Fab fragment, a F(ab′)2 fragment, a Fab′ fragment, an scFv fragment, an scFv-Fc fragment, and/or a single-domain antibody or antigen binding fragment thereof. In some embodiments, the collagen-targeted constructs comprise an Fc domain that has reduced or no effector function.

In some embodiments, the antibody or antigen-binding fragment thereof is fully human, humanized, and/or chimeric.

In some embodiments, the collagen-targeted construct is a fusion protein.

In some embodiments, (i) and (ii) as described above are joined via chemical conjugation.

In some embodiments, the collagen VII-targeted construct is a multispecific construct.

In some embodiments, the collagen VII-targeted construct comprises (i) more than one (e.g., two, three, four, or five) collagen VII-binding domains and/or (ii) more than one (e.g., two, three, four, or five) inflammatory cytokine binding domain(s).

Also provided herein are multispecific antibodies that include (a) first antigen-binding domain that specifically binds to human collagen VII and (b) a second antigen-binding domain that (i) binds to an inflammatory cytokine and (ii) inhibits binding of the inflammatory cytokine to its cognate receptor.

In some embodiments, the multispecific antibody is a bispecific antibody.

In some embodiments, the second antigen-binding domain binds to an inflammatory cytokine selected from the group consisting of TNFα, IL-17A, IL-12, IL-23, IL-6, IL-4, and IFNγ.

In some embodiments, any of the collagen-targeted constructs (including multispecific antibodies) described herein comprise an Fc region of an immunoglobulin. In some embodiments, the Fc region has reduced or no effector function. For example, in some embodiments, the Fc region is an altered Fc constant region containing one or more amino acid substitutions, insertions, or deletions, relative to a wild-type Fc region of the isotype.

Also provided herein are nucleic acids encoding the collagen-targeted constructs or the multispecific antibodies described herein. In addition, the disclosure also features vectors or expression vectors comprising the nucleic acids. In another aspect, the disclosure features a cell (e.g., a mammalian host cell) comprising the nucleic acid, vector, or expression vector. In yet another aspect, the disclosure features a method for producing a collagen VII-targeted construct described herein, which method comprises culturing the aforementioned cell (or a population of such cells) under conditions conducive for expression of the construct by the cell. The method can further include isolating the expressed construct from either the cells or the culture medium in which the cell or cells were cultured.

Also provided herein are methods for reducing inflammation in the gut, lung, or skin of a subject, comprising administering a therapeutically effective amount of a collagen-targeted construct, a nucleic acid, or a multispecific antibody as described herein, to a subject in need thereof.

In another aspect, the disclosure features a method of treating a subject who has a disorder associated with an inflammatory response in a tissue that expresses collagen VII, which method comprises administering a therapeutically effective amount of a collagen-targeted construct, a nucleic acid, or a multispecific antibody, to a subject in need thereof.

In some embodiments, the tissue is the lung, skin, or gut of the subject.

In some embodiments, the subject has rheumatoid arthritis, psoriasis, inflammatory bowel disease, asthma, atopic dermatitis, dermatomyositis, systemic or cutaneous lupus erythematosus, scleroderma, graft-versus-host disease, or organ transplant rejection after transplant of an organ that includes an epithelial layer.

In another aspect, the disclosure features a method of reducing immune cell infiltration at a site of inflammation in a subject, preferably wherein the site of inflammation is in the gut, skin, or lungs of the subject, the method comprising administering a therapeutically effective amount of a collagen-targeted construct, a nucleic acid, or a multispecific antibody, to a subject in need thereof.

In another aspect, the disclosure features a method of inhibiting inflammatory cytokine activity (which activity can include production of other cytokines by immune cells stimulated with one of the inflammatory cytokines) at a site of inflammation in a subject, preferably wherein the site of inflammation is in the gut, skin, or lungs of the subject, the method comprising administering a therapeutically effective amount of a collagen-targeted construct, a nucleic acid, or a multispecific antibody, to a subject in need thereof.

In another aspect, the disclosure features a method of treating skin inflammation in a subject, the method comprising administering a therapeutically effective amount of a collagen-targeted construct, a nucleic acid, or a multispecific antibody, to the skin of a subject in need thereof. In some embodiments, the subject has psoriasis, atopic dermatitis, dermatomyositis, systemic or cutaneous lupus erythematosus, scleroderma, graft-versus-host disease, or organ transplant rejection after a skin transplant.

In another aspect, the disclosure features a method of treating lung inflammation in a subject, the method comprising administering a therapeutically effective amount of a collagen-targeted construct, a nucleic acid, or a multispecific antibody, to the lungs of a subject in need thereof. In some embodiments, the subject has asthma, graft-versus-host disease, or organ transplant rejection after transplant of a lung.

In another aspect, the disclosure features a method of treating gut inflammation in a subject, the method comprising administering a therapeutically effective amount of a collagen-targeted construct, a nucleic acid, or a multispecific antibody, to the gut of a subject in need thereof. In some embodiments, the subject has inflammatory bowel disease.

As used herein, the term “bispecific” or “bifunctional antibody” refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, the term “kd” is intended to refer to the off rate constant for the dissociation of an antibody from the antibody/antigen complex.

As used herein, the term “ka” is intended to refer to the on rate constant for the association of an antibody with the antigen.

The equilibrium constant KD is the ratio of the kinetic rate constants—kd/ka.

As used herein, the terms “polypeptide,” “peptide”, and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “purified” or “isolated” as applied to any of the proteins (antibodies or fragments) described herein refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

As used herein, the terms “inhibits” or “blocks” (e.g., referring to inhibition/blocking of binding of a human cytokine to its cognate receptor on cells, or inhibiting/blocking of dimerization of cytokine, are used interchangeably and encompass both partial and complete inhibition/blocking. The inhibition/blocking of reduces or alters the normal level or type of activity that occurs when an inflammatory cytokine binds to its cognate receptor that occurs without inhibition or blocking. Inhibition and blocking are also intended to include any measurable decrease in the binding affinity of an inflammatory cytokine when in contact with an antigen-binding domain as compared to the cytokine not in contact with the binding domain, e.g., inhibits binding of the inflammatory cytokine by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

With regard to the binding of a binding domain to a target molecule, the terms “specific binding,” “specifically binds to,” “specific for,” “selectively binds,” and “selective for” a particular antigen (e.g., a polypeptide target) or an epitope on a particular antigen mean binding that is measurably different (i.e., the KD is at least 5 fold lower, e.g., has at least 10, 20, 50, 100, 500, or 1000-fold or more higher affinity) from a non-specific or non-selective interaction. In some embodiments, an antigen-binding domain (e.g., an antibody or antigen-binding fragment thereof) specific for human collagen VII does not detectably bind to other human collagen isoforms, e.g., does not bind to Collagen I, II, III, IV, V, VI or VIII to XXVIII (see Ricard-Blum et al., Cold Spring Harb Perspect Biol. 2011 January; 3(1): a004978). In some embodiments, an antigen-binding domain (e.g., an antibody or antigen-binding fragment thereof) specific for human collagen VII has at least a 5-fold (e.g., at least a 10, 20, 25, 30, 50, 75, 100, 200, or 500-fold or more) greater affinity for human collagen VII relative to its affinity human proteins that are less than 95% identical to the full length human Collagen VII sequence, i.e., amino acids 17-2944 of SEQ ID NO:1. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

As used herein, the term “subject” means a mammalian subject. Exemplary subjects include, but are not limited to humans, monkeys, dogs, cats, mice, rats, cows, horses, camels, goats and sheep. In s embodiments, the subject is a human. In some embodiments, the subject has or is suspected to have a disease or condition that can be treated with a therapeutic construct provided herein. In some aspects, the disease or condition is an autoimmune disease or an inflammatory condition, such as any of those described herein or known in the art.

As used herein, the term “patient” includes human and other mammalian subjects that receive either prophylactic or therapeutic treatment.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules that are present in the natural source of the nucleic acid. For example, in various embodiments, the isolated nucleic acid molecule can contain less than about 0.1 kb of 5′ and/or 3′ untranslated nucleotide sequences which naturally flank the nucleic acid molecule, e.g., in the mRNA. Moreover, an “isolated” nucleic acid molecule, such as a cDNA molecule, is substantially free of other cellular material, or culture medium when produced by recombinant techniques, or substantially free of chemical precursors or other chemicals when chemically synthesized.

To determine the percent identity of two amino acid or nucleic acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic illustration of the major organs with epithelial-associated collagen VII expression in the human body, i.e., the skin, lung, and gut.

FIG. 1B is a schematic illustration of an exemplary construct described herein. On the left is a collagen-VII binding domain, e.g., all or part of an anti-Collagen VII antibody. The circle in the center represents a linkage, which may be direct, covalent, chemical linkage, or via a peptide or chemical linker. On the right is the cytokine binding domain, e.g., all or part of an anti-cytokine antibody.

FIG. 2A is a schematic illustration of a non-targeted anti-cytokine antibody, e.g., anti-TNFα antibody, conjugated to biotin.

FIG. 2B is a schematic illustration of an exemplary targeted construct, wherein an anti-TNFα antibody is conjugated to an anti-Collagen VII antibody via biotin-streptavidin reaction.

FIG. 3 is a schematic illustration of a pilot study in a human engrafted mouse model of graft versus host disease (GvHD)-like skin inflammation. A subtherapeutic dose of a TNF receptor/IgG1 constant region fusion protein (etanercept):biotin (20 μg, equivalent to about ⅕ the standard therapeutic dose) or the collagen VII-targeted conjugate (containing the same molar dose of the TNFR/IgG1 Fc) was injected on days 6 and 10 after PBMC infusion.

FIGS. 4A-B show that a subtherapeutic (20 μg) dose of collagen VII-targeted anti-TNFα (etanercept) effectively suppressed skin inflammation. 4A, Histologic inflammation including epithelial injury, keratinocyte dyskeratosis, dermal inflammation and T cell infiltration was suppressed by collagen VII-targeted anti-TNFα but not by anti-TNFα alone. The collagen VII targeting provided effective suppression of inflammation at ⅕ of the standard therapeutic dose. B) four bar graphs showing that collagen VII-targeted anti-TNFα decreased the production of inflammatory cytokines, as measured by quantitative RT-PCR (results shown are normalized to CD3).

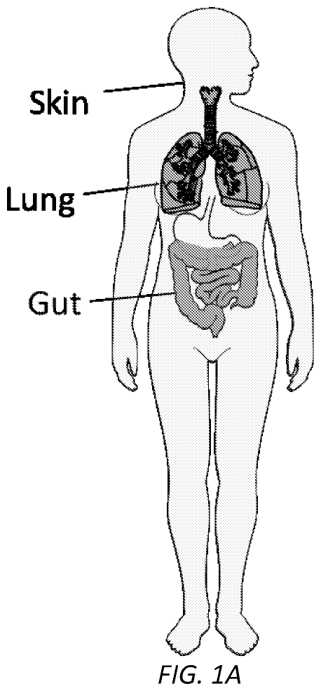

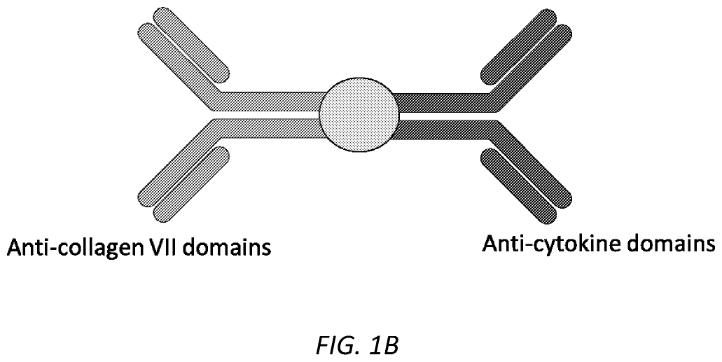

### DETAILED DESCRIPTION

Recent research suggests that much of the pathologic inflammation associated with human inflammatory and autoimmune diseases occurs within the target tissues themselves, not within lymph nodes (see, e.g., Clark, R. A. (2015). Sci Transl Med 7(269): 269rv261). Thus, if biologic medications could be specifically targeted to the relevant affected tissue (e.g. the skin in psoriasis, the gut in inflammatory bowel disease and the joints in rheumatoid arthritis), then the local pathologic inflammation could be suppressed without suppressing immunity in 1) other tissues and 2) in secondary lymphoid organs such as the lymph nodes and spleen. Described herein are methods and compositions that target biologic molecules by taking advantage of the fact that peripheral tissues have distinct types of collagen. For example, collagen VII forms anchoring fibrils that help to tether the epidermis to the sub-epidermal tissues in skin, gut and lung (see Sakai et al., J Cell Biol. 1986; 103:1577-86). Described herein are collagen-targeted constructs that include anti-inflammatory biologics linked to collagen VII-binding domains that are useful for inflammatory diseases in skin, gut and lung. These collagen-targeted biologics could be used at much lower doses than conventional biologics, would preferentially accumulate in the affected tissues, and would likely markedly reduce or eliminate entirely the risk of infections. This would reduce infectious complications in patients with severe disease who would otherwise receive conventional, non-targeted biologics. Moreover, the improved safety profile of these medications would make it possible to extend their use to the majority of patients with inflammatory diseases, not just the limited number of patients in whom severe disease balances the risk of infectious complications. Because these tissue targeted biologics will deposit in the extracellular matrix of the affected tissues, they may have an extended half-life and lead to longer periods of disease remission.

The collagen-targeted constructs described herein can be, e.g., fusion proteins that are encoded by a single nucleic acid, or they can be made by conjugating two or more separate proteins together. The collagen VII-binding domain and the cytokine binding domain(s) are linked to each other, and each binding domain is capable of binding its respective antigen or receptor at the same time as each of the other antigen binding domains.

### Binding Domains

As noted above, the present constructs include: (i) one or more (e.g., one, two, three, four, or five) collagen VII-binding domains and (ii) one or more (e.g., one, two, three, four, or five) inflammatory cytokine binding domain(s). Each of these binding domains can independently be, or include, for example: an antibody or antigen-binding fragment thereof, a soluble form of a protein (e.g., a soluble form of a receptor for an inflammatory cytokine), or a non-antibody scaffold protein, each of which is known in the art and described herein.

### Antibodies

In some embodiments, one or both of the binding domains as described herein is an antibody or antigen-binding fragment thereof. As used herein, the term “antibody” refers to a whole antibody comprising two light chain polypeptides and two heavy chain polypeptides. Whole antibodies include different antibody isotypes including IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

As used herein, the term “antibody fragment,” “antigen-binding fragment,” or similar terms refer to a fragment of an antibody that retains the ability to bind to a target antigen (e.g., collagen VII) and promote, induce, and/or increase the activity of the target antigen. Such fragments include, e.g., a single chain antibody, a single chain Fv fragment (scFv), an Fd fragment, an Fab fragment, an Fab′ fragment, or an F(ab′)₂ fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12):1121-1123; Rondon and Marasco (1997) Annual Review of Microbiology 51:257-283, the disclosures of each of which are incorporated herein by reference in their entirety.

In some embodiments, the antibody fragment described herein is a nanobody, such as a camelid or dromedary antibodies (e.g., antibodies derived from Camelus bactrianus, Calelus dromaderius, or Lama paccos). Such antibodies, unlike the typical two-chain (fragment) or four-chain (whole antibody) antibodies from most mammals, generally lack light chains. See U.S. Pat. No. 5,759,808; Stijlemans et al. (2004) J Biol Chem 279:1256-1261; Dumoulin et al. (2003) Nature 424:783-788; and Pleschberger et al. (2003) Bioconjugate Chem 14:440-448. As with other antibodies of non-human origin, an amino acid sequence of a camelid antibody can be altered recombinantly to obtain a sequence that more closely resembles a human sequence, i.e., the nanobody can be “humanized” to thereby further reduce the potential immunogenicity of the antibody. The antibody can be a polyclonal, monoclonal, recombinant, e.g., a chimeric, de-immunized or humanized, fully human, non-human, e.g., murine, or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., are IgG4 antibodies that lack effector function or have a mutagenized or deleted Fc receptor binding region; substitutions in human IgG1 of IgG2 residues at positions 233-236 and IgG4 residues at positions 327, 330 and 331 were shown to greatly reduce ADCC and CDC (see, e.g., Armour et al., 1999. Eur J Immunol. 29(8):2613-24; Shields et al., 2001. J Biol Chem. 276(9):6591-604), and alanine substitution at positions including K322 significantly reduce complement activation (Idusogie et al., 2000. J Immunol. 164(8):4178-84); see also US20150337053 and references cited therein. The antibody can be coupled to a toxin or imaging agent.

Methods for making suitable antibodies are known in the art. A full-length antigen or antigenic peptide fragment thereof can be used as an immunogen, or can be used to identify antibodies made with other immunogens, e.g., cells, membrane preparations, and the like, e.g., E rosette positive purified normal human peripheral T cells, as described in U.S. Pat. Nos. 4,361,549 and 4,654,210.

Methods for making monoclonal antibodies are known in the art. Basically, the process involves obtaining antibody-secreting immune cells (lymphocytes) from the spleen of a mammal (e.g., mouse) that has been previously immunized with the antigen of interest either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Mammalian lymphocytes are immunized by in vivo immunization of the animal (e.g., a mouse) with the desired antigen. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511 (1976), which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in E. Harlow, et. al., editors, Antibodies: A Laboratory Manual (1988).

Fully human antibodies are also provided in the disclosure. The term “human antibody” includes antibodies having variable and constant regions (if present) derived from human immunoglobulin sequences, preferably human germline sequences. Human antibodies can include amino acid residues not encoded by human germline immunoglobulin sequences (e.g., mutations introduced by random or site-specific mutagenesis in vitro or by somatic mutation in vivo). However, the term “human antibody” does not include antibodies in which CDR sequences derived from another mammalian species, such as a mouse, have been grafted onto human framework sequences (i.e., humanized antibodies). Fully human or human antibodies may be derived from transgenic mice carrying human antibody genes (carrying the variable (V), diversity (D), joining (J), and constant (C) exons) or from human cells. For example, it is now possible to produce transgenic animals (e.g., mice) that are capable, upon immunization, of producing a full repertoire of human antibodies in the absence of endogenous immunoglobulin production. See, e.g., Jakobovits et al. (1993) Proc Natl Acad Sci USA 90:2551; Jakobovits et al. (1993) Nature 362:255-258; Bruggemann et al. (1993) Year in Immunol. 7:33; and Duchosal et al. (1992) Nature 355:258. Transgenic mouse strains can be engineered to contain gene sequences from unrearranged human immunoglobulin genes. One example of such a mouse is the HuMAb Mouse® (Medarex, Inc.), which contains human immunoglobulin transgene miniloci that encode unrearranged human μ heavy and κ light chain immunoglobulin sequences, together with targeted mutations that inactivate the endogenous μ and κ chain loci. See, e.g., Lonberg, et al. (1994) Nature 368(6474):856-859. The preparation and use of HuMab mice, and the genomic modifications carried by such mice, are further described in Taylor et al. (1992) Nucleic Acids Res 20:6287-6295; Chen, J. et al. (1993) International Immunology 5: 647-656; Tuaillon et al. (1993) Proc Natl Acad Sci USA 90:3720-3724; Choi et al. (1993) Nature Genetics 4:1 17-123; Tuaillon et al. (1994) J Immunol 152:2912-2920; Taylor et al. (1994) International Immunology 6:579-591; and Fishwild et al. (1996) Nature Biotechnol 14:845-851. An alternative transgenic mouse system for expressing human immunoglobulin genes is referred to as the Xenomouse (Abgenix, Inc.) and is described in, e.g., U.S. Pat. Nos. 6,075,181; 6,114,598; 6,150,584; and 6,162,963. Like the HuMAb Mouse® system, the Xenomouse system involves disruption of the endogenous mouse heavy and light chain genes and insertion into the genome of the mouse transgenes carrying unrearranged human heavy and light chain immunoglobulin loci that contain human variable and constant region sequences. Other systems known in the art for expressing human immunoglobulin genes include the KM Mouse® system, described in detail in PCT Publication WO 02/43478 and the TC mouse system described in Tomizuka et al. (2000) Proc Natl Acad Sci USA 97:722-727.

The human sequences may code for both the heavy and light chains of human antibodies and would function correctly in the mice, undergoing rearrangement to provide a wide antibody repertoire similar to that in humans. The transgenic mice can be immunized with the target protein immunogen to create a diverse array of specific antibodies and their encoding RNA. Nucleic acids encoding the antibody chain components of such antibodies may then be cloned from the animal into a display vector. Typically, separate populations of nucleic acids encoding heavy and light chain sequences are cloned, and the separate populations then recombined on insertion into the vector, such that any given copy of the vector receives a random combination of a heavy and a light chain. The vector is designed to express antibody chains so that they can be assembled and displayed on the outer surface of a display package containing the vector. For example, antibody chains can be expressed as fusion proteins with a phage coat protein from the outer surface of the phage. Thereafter, display packages can be selected and screened for display of antibodies binding to a target.

In some embodiments, a skilled artisan can identify an antibody from a non-immune biased library as described in, e.g., U.S. Pat. No. 6,300,064 (to Knappik et al.; Morphosys AG) and Schoonbroodt et al. (2005) Nucleic Acids Res 33(9):e81.

In some embodiments, the methods described herein can involve, or be used in conjunction with, e.g., phage display technologies, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) Biotechnol Prog 18:212-220; Boder et al. (2000) Methods Enzymology 328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999) J Immunol Methods 231:119-135; and Hanes et al. (2000) Nat Biotechnol 18:1287-1292).

In some embodiments, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995), supra; Ames et al. (1995), supra; Kettleborough et al. (1994), supra; Persic et al. (1997), supra; and Burton et al. (1994), supra. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some embodiments, require use of a helper phage. In some embodiments, no helper phage is required (see, e.g., Chasteen et al. (2006) Nucleic Acids Res 34(21):e145). Phage produced from the bacteria are recovered and then contacted to, e.g., a target antigen bound to a solid support (immobilized). Phage may also be contacted to antigen in solution, and the complex is subsequently bound to a solid support.

In some embodiments, the immobilized phage are the phage of interest. Accordingly, the unbound phage are removed by washing the support. Following the wash step, bound phage are then eluted from the solid support, e.g., using a low pH buffer or a free target antigen competitor, and recovered by infecting bacteria. In some embodiments, the phage that are not immobilized are the phage of interest. In such embodiments, the population of phage can be contacted to the antigen two or more times to deplete from the population any of the phage that bind to the support. Unbound phage are then collected and used for subsequent screening steps.

To enrich the phage population for phage particles that contain antibodies having a higher affinity for the target antigen (while reducing the proportion of phage that may bind to the antigen non-specifically), the eluted phage (described above) can be used to re-infect a population of bacterial host cells. The expressed phage are then isolated from the bacteria and again contacted to a target antigen. The concentration of antigen, pH, temperature and inclusion of detergents and adjuvants during contact can be modulated to enrich for higher affinity antibody fragments. The unbound phage are removed by washing the solid support. The number or cycles, duration, pH, temperature and inclusion of detergents and adjuvants during washing can also be modulated to enrich for higher affinity antibody fragments. Following the wash step, bound phage are then eluted from the solid support. Anywhere from one to six iterative cycles of panning may be used to enrich for phage containing antibodies having higher affinity for the target antigen. In some embodiments, a deselection step can also be performed in conjunction with any of the panning approaches described herein.

Individual phage of the population can be isolated by infecting bacteria and then plating at a density to allow formation of monoclonal antibodies.

Alternatively or in addition, phage-displayed synthetic antibody libraries built on a single framework with diversity restricted to four complementarity-determining regions by using precisely designed degenerate oligonucleotides can be used; see, Chen and Sidhu, Methods Mol Biol. 2014; 1131:113-31. Codon-precise, synthetic, antibody fragment libraries built using automated hexamer codon additions can also be used, see Frigotto et al., Antibodies 2015, 4, 88-102.

A subpopulation of antibodies screened using the above methods can be characterized for their specificity and binding affinity for a particular antigen (e.g., human collagen VII) using any immunological or biochemical based method known in the art. For example, specific binding of an antibody, may be determined for example using immunological or biochemical based methods such as, but not limited to, an ELISA assay, SPR assays, immunoprecipitation assay, affinity chromatography, and equilibrium dialysis as described above. Immunoassays which can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

In addition to utilizing whole antibodies, the invention encompasses the use of binding portions of such antibodies. Such binding portions include Fab fragments, F(ab′)2 fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983).

Chimeric, humanized, de-immunized, or completely human antibodies are desirable for applications which include repeated administration, e.g., therapeutic treatment of human subjects.

Chimeric antibodies generally contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and 0i, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target protein. In some embodiments, the antibody is monovalent, e.g., as described in Abbs et al., Ther. Immunol. 1(6):325-31 (1994), incorporated herein by reference.

In some embodiments, the constructs described herein are multi specific or bispecific antibodies. As used herein, the term “bispecific” or “bifunctional antibody” refers to an artificial hybrid antibody having two different heavy/light chain pairs and two different binding sites; a multi specific antibody has more than two (e.g., at least three or four) different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148:1547-1553 (1992); Spiess et al., Molecular Immunology 67:95-106 (2015); Kontermann and Brinkmann, Drug Discovery Today 20(7):838-847 (2015).

Traditionally, the recombinant production of bispecific antibodies is based on the co-expression of two immunoglobulin heavy-chain/light-chain pairs, where the two heavy chain/light-chain pairs have different specificities (Milstein and Cuello (1983) Nature 305:537-539). Antibody variable domains with the desired binding specificities (antibody-antigen combining sites) can be fused to immunoglobulin constant domain sequences. The fusion of the heavy chain variable region can be, e.g., with an immunoglobulin heavy-chain constant domain, including at least part of the hinge, CH2, and CH3 regions. For further details of illustrative currently known methods for generating bispecific antibodies see, e.g., Suresh et al. (1986) Methods in Enzymology 121:210; PCT Publication No. WO 96/27011; Brennan et al. (1985) Science 229:81; Shalaby et al., J Exp Med (1992) 175:217-225; Kostelny et al. (1992) J Immunol 148(5):1547-1553; Hollinger et al. (1993) Proc Natl Acad Sci USA 90:6444-6448; Gruber et al. (1994) J Immunol 152:5368; Spiess et al., Molecular Immunology 67:95-106 (2015); Kontermann and Brinkmann, Drug Discovery Today 20(7):838-847 (2015); and Tutt et al. (1991) J Immunol 147:60. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148(5):1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368. Alternatively, the antibodies can be “linear antibodies” as described in, e.g., Zapata et al. (1995) Protein Eng. 8(10):1057-1062. Briefly, these antibodies comprise a pair of tandem Fd segments (VH-CH1-VH-CH1) which form a pair of antigen binding regions. Linear antibodies can be bispecific or monospecific.

Antibodies with more than two valencies (e.g., trispecific antibodies) are contemplated and described in, e.g., Tutt et al. (1991) J Immunol 147:60.

The disclosure also embraces variant forms of multi-specific antibodies such as the dual variable domain immunoglobulin (DVD-Ig) molecules described in Wu et al. (2007) Nat Biotechnol 25(11): 1290-1297. The DVD-Ig molecules are designed such that two different light chain variable domains (VL) from two different parent antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region. Methods for making DVD-Ig molecules from two parent antibodies are further described in, e.g., PCT Publication Nos. WO 08/024188 and WO 07/024715. In some embodiments, the bispecific antibody is a Fabs-in-Tandem immunoglobulin, in which the light chain variable region with a second specificity is fused to the heavy chain variable region of a whole antibody. Such antibodies are described in, e.g., International Patent Application Publication No. WO 2015/103072.

Also provided herein are tetravalent antibodies with two binding sites for each antigen (e.g., two collagen binding domains and two cytokine binding domains) created by the fusion of a second binding moiety, for example a single-chain Fv fragment or a domain antibody to the N or C terminus of the heavy or light chain, respectively, of an antibody. Other possible forms include Triomabs, kih IgG with common LC, CrossMab, ortho-Fab IgG, 2-in-1-IgG, IgG-scFv, ScFv2-Fc, bi-nanobody, BiTE (scFvs that are connected by flexible linker peptides), tandAbs (bispecific fusion proteins with four binding sites that do not include Fc domains), dual affinity re-targeting (DART) constructs that are diabody-like entities that have the VH of a first variable region linked to the VL of the second binder, and the VH of a second variable region linked to the VL of the first; DART-Fc; scFv-HAS-scFv (scFvs linked to the constant region of an IgG, contains four binding regions, two for each specificity); see Kontermann and Brinkmann, Drug Discovery Today 20(7):838-847 (2015) and references cited therein, and Spiess et al., Molecular Immunology 67 (2015) 95-106 and references cited therein.

In some embodiments, the antibodies described herein comprise an altered heavy chain constant region that has reduced (or no) effector function relative to its corresponding unaltered constant region. Effector functions involving the constant region of the antibody may be modulated by altering properties of the constant or Fc region. Altered effector functions include, for example, a modulation in one or more of the following activities: antibody-dependent cellular cytotoxicity (ADCC), complement-dependent cytotoxicity (CDC), apoptosis, binding to one or more Fc-receptors, and pro-inflammatory responses. Modulation refers to an increase, decrease, or elimination of an effector function activity exhibited by a subject antibody containing an altered constant region as compared to the activity of the unaltered form of the constant region. In particular embodiments, modulation includes situations in which an activity is abolished or completely absent.

An altered constant region with altered FcR binding affinity and/or ADCC activity and/or altered CDC activity is a polypeptide which has either an enhanced or diminished FcR binding activity and/or ADCC activity and/or CDC activity compared to the unaltered form of the constant region. An altered constant region which displays increased binding to an FcR binds at least one FcR with greater affinity than the unaltered polypeptide. An altered constant region which displays decreased binding to an FcR binds at least one FcR with lower affinity than the unaltered form of the constant region. Such variants which display decreased binding to an FcR may possess little or no appreciable binding to an FcR, e.g., 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the binding to the FcR as compared to the level of binding of a native sequence immunoglobulin constant or Fc region to the FcR. Similarly, an altered constant region that displays modulated ADCC and/or CDC activity may exhibit either increased or reduced ADCC and/or CDC activity compared to the unaltered constant region. For example, in some embodiments, the antibody comprising an altered constant region can exhibit approximately 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the ADCC and/or CDC activity of the unaltered form of the constant region. An antibody described herein comprising an altered constant region displaying reduced ADCC and/or CDC may exhibit reduced or no ADCC and/or CDC activity.

In some embodiments, an antibody described herein exhibits reduced or no effector function. In some embodiments, an antibody comprises a hybrid constant region, or a portion thereof, such as a G2/G4 hybrid constant region (see e.g., Burton et al. (1992) Adv Immun 51:1-18; Canfield et al. (1991) J Exp Med 173:1483-1491; and Mueller et al. (1997) Mol Immunol 34(6):441-452).

In some embodiments the constructs lack all of ADCC, CDC, ability to induce apoptosis, binding to Fc-receptors, and ability to initiate pro-inflammatory responses.

### Non-Antibody Binding Domains

In some embodiments, a binding domain described herein is a non-antibody, scaffold protein. These proteins are, generally, obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. Ali et al. (1999) J Biol Chem 274:24066-24073. The portion of human transferrin not involved with binding the receptor remains unchanged and serves as a scaffold, like framework regions of antibodies, to present the variant binding sites. The libraries are then screened, as an antibody library is, and in accordance with the methods described herein, against a target antigen of interest to identify those variants having optimal selectivity and affinity for the target antigen. Hey et al. (2005) TRENDS Biotechnol 23(10):514-522.

One of skill in the art would appreciate that the scaffold portion of the non-antibody scaffold protein can include, e.g., all or part of: the Z domain of S. aureus protein A, human transferrin, human tenth fibronectin type III domain, kunitz domain of a human trypsin inhibitor, human CTLA-4, an ankyrin repeat protein, a human lipocalin (e.g., anticalins), human crystallin, human ubiquitin, or a trypsin inhibitor from E. elaterium. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody), ankyrin repeats (e.g., DARPins), gamma-B-crystallin/ubiquitin (e.g., Affilins), CTLD₃ (e.g., Tetranectins), and (LDLR-A module) (e.g., Avimers). Additional information on alternative scaffolds is provided in Binz et al., Nat. Biotechnol., 2005 23:1257-1268; and Skerra, Current Opin. in Biotech., 2007 18:295-304, each of which is incorporated by reference in its entirety.

### Collagen VII

The compositions described herein include a collagen-binding domain. The binding domain is preferably specific for a certain collagen isoform, e.g., for Collagen VII, i.e., binds to Collagen VII, and does not show substantial binding (i.e., the KD is at least 2 fold, e.g., at least 5, 10. 20, 50, or 100-fold different) to other proteins or other isoforms of Collagen.

Collagen VII is composed of three identical alpha collagen chains and expression of Collagen VII is generally restricted to the basement zone beneath stratified squamous epithelia of the gut, lungs, and skin, where it acts as an anchor tying the external epithelial layer to the underlying stromal layer. See, e.g., Wetzels et al., Histopathology 1992 April; 20(4):295-303;

An exemplary sequence for the alpha chain of Collagen VII, also referred to as collagen alpha-1 (VII) chain precursor, is shown in SEQ ID NO:1:

```
SEQ ID NO: 1
      1 mtlrllvaal cagilaeapr vraqhrervt ctrlyaadiv flldgsssig rsnfrevrsf
    61 leglvlpfsg aasaqgvrfa tvqysddprt efgldalgsg gdvirairel sykggntrtg
  121 aailhvadhv flpqlarpgv pkvcilitdg ksqdlvdtaa qrlkgqgvkl favgiknadp
  181 eelkrvasqp tsdffffvnd fsilrtllpl vsrrvcttag gvpvtrppdd stsaprdlvl
  241 sepssgslrv qwtaasgpvt gykvqytplt glgqplpser qevnvpaget svrlrglrpl
  301 teyqvtvial yansigeavs gtarttaleg peltiqntta hsllvawrsv pgatgyrvtw
  361 rvlsggptqq gelgpgqgsv llrdlepgtd yevtvstlfg rsvgpatslm artdasveqt
  421 lrpvilgpts illswnlvpe argyrlewrr etgleppqkv vlpsdvtryq ldglqpgtey
  481 rltlytlleg hevatpatvv ptgpelpvsp vtdlqatelp gqrvrvswsp vpgatqyrii
  541 vrstqgvert lvlpgsqtaf dlddvqagls ytvrvsarvg pregsasvlt vrrepetpla
  601 vpglrvvvsd atrvrvawgp vpgasgfris wstgsgpess qtlppdstat ditglqpgtt
  661 yqvavsvlrg reegpaaviv artdplgpvr tvhvtqasss svtitwtrvp gatgyrvswh
  721 sahgpeksql vsgeatvael dglepdteyt vhvrahvagv dgppasvvvr tapepvgrvs
  781 rlqilnassd vlritwvgvt gatayrlawg rseggpmrhq ilpgntdsae irgleggvsy
  841 svrvtalvgd regtpvsivv ttppeappal gtlhvvqrge hslrlrwepv praqgfllhw
  901 qpeggqegsr vlgpelssyh ldglepatqy rvrlsvlgpa gegpsaevta rtesprvpsi
  961 elrvvdtsid svtlawtpvs rassyilswr plrgpgqevp gspqtlpgis ssqrvtglep
1021 gvsyifsltp vldgvrgpea svtqtpvcpr gladvvflph atqdnahrae atrrvlerlv
1081 lalgplgpqa vqvgllsysh rpsplfplng shdlgiilqr irdmpymdps gnnlgtavvt
1141 ahrymlapda pgrrqhvpgv mvllvdeplr gdifspirea gasglnvvml gmagadpeql
1201 rrlapgmdsv qtffavddgp sldqaysgla talcgasftt qprpepcpvy cpkgqkgepg
1261 emglrgqvgp pgdpglpgrt gapgpqgppg satakgergf pgadgrpgsp gragnpgtpg
1321 apglkgspgl pgprgdpger gprgpkgepg apgqviggeg pglpgrkgdp gpsgppgprg
1381 plgdpgprgp pglpgtamkg dkgdrgergp pgpgeggiap gepglpglpg spgpqgpvgp
1441 pgkkgekgds edgapglpgq pgspgeqgpr gppgaigpkg drgfpgplge agekgergpp
1501 gpagsrglpg vagrpgakgp egppgptgrq gekgepgrpg dpavvgpava gpkgekgdvg
1561 pagprgatgv qgergppglv lpgdpgpkgd pgdrgpiglt gragppgdsg ppgekgdpgr
1621 pgppgpvgpr grdgevgekg degppgdpgl pgkagerglr gapgvrgpvg ekgdqgdpge
1681 dgrngspgss gpkgdrgepg ppgppgrlvd tgpgarekge pgdrgqegpr gpkgdpglpg
1741 apgergiegf rgppgpqgdp gvrgpagekg drgppgldgr sgldgkpgaa gpsgpngaag
1801 kagdpgrdgl pglrgegglp gpsgppglpg kpgedgkpgl ngkngepgdp gedgrkgekg
1861 dsgasgregr dgpkgergap gilgpqgppg lpgpvgppgq gfpgvpggtg pkgdrgetgs
1921 kgeqglpger glrgepgsvp nvdrlletag ikasalreiv etwdessgsf lpvperrrgp
1981 kgdsgeqgpp gkegpigfpg erglkgdrgd pgpqgppgla lgergppgps glagepgkpg
2041 ipglpgragg vgeagrpger gergekgerg eggrdgppgl pgtpgppgpp gpkvsvdepg
2101 pglsgeggpp glkgakgepg sngdqgpkgd rgvpgikgdr gepgprgqdg npglpgergm
2161 agpegkpglq gprgppgpvg ghgdpgppga pglagpagpq gpsglkgepg etgppgrglt
2221 gptgavglpg ppgpsglvgp qgspglpgqv getgkpgapg rdgasgkdgd rgspgvpgsp
2281 glpgpvgpkg epgptgapgq avvglpgakg ekgapgglag dlvgepgakg drglpgprge
2341 kgeagragep gdpgedgqkg apgpkgfkgd pgvgvpgspg ppgppgvkgd lglpglpgap
2401 gvvgfpgqtg prgemgqpgp sgerglagpp gregipgplg ppgppgsvgp pgasglkgdk
2461 gdpgvglpgp rgergepgir gedgrpgqeg prgltgppgs rgergekgdv gsaglkgdkg
2521 dsavilgppg prgakgdmge rgprgldgdk gprgdngdpg dkgskgepgd kgsaglpglr
2581 gllgpqgqpg aagipgdpgs pgkdgvpgir gekgdvgfmg prglkgergv kgacgldgek
2641 gdkgeagppg rpglaghkge mgepgvpgqs gapgkeglig pkgdrgfdgq pgpkgdqgek
2701 gergtpgigg fpgpsgndgs agppgppgsv gprgpeglqg qkgergppge rvvgapgvpg
2761 apgergeqgr pgpagprgek geaalteddi rgfvrqemsq hcacqgqfia sgsrplpsya
2821 adtagsqlha vpvlrvshae eeervppedd eyseyseysv eeyqdpeapw dsddpcslpl
2881 degsctaytl rwyhravtgs teachpfvyg gcggnanrfg treacerrcp prvvqsqgtg
2941 taqd
```

The mature Collagen VII peptide is amino acids 17-2944 of SEQ ID NO:1. An exemplary nucleic acid sequence encoding human Collagen VII is in GenBank at Acc. No. NM_000094.3, and the genomic sequence for the human COL7A1 gene (which has 118 exons) is at RefSeqGene ID NG 007065.1. See also Parente et al., Proceedings of the National Academy of Sciences of the United States of America. 88 (16): 6931-5 (1991).

A Collagen VII binding domain can include an antibody or antigen-binding fragment thereof as known in the art and described herein. In some embodiments, the binding domain binds to the Collagen VII protein but does not affect its function, i.e., does not significantly disrupt or diminish binding to laminin 5 or fibronectin. A number of collagen VII-binding antibodies are known in the art and commercially available, e.g., from Abbexa Ltd; Abcam; Acris Antibodies GmbH; AMSBIO LLC; antibodies-online; Atlas Antibodies; Aviva Systems Biology; Bio-Rad (Formerly AbD Serotec); Biorbyt; Bioss Inc.; Bosterbio; Cloud-Clone; Creative Diagnostics; Fitzgerald Industries International; GeneTex; GenWay Biotech, Inc.; Invitrogen Antibodies; LifeSpan BioSciences; MyBioSource.com; Novus Biologicals; ProSci, Inc; Raybiotech, Inc.; Santa Cruz Biotechnology, Inc.; Signalway Antibody LLC; St John's Laboratory; and United States Biological. See also Woodley et al., J Invest Dermatol. 2005 May; 124(5):958-64; Sakai et al., J Cell Biol. 1986 October; 103(4):1577-86; and Tanaka et al., Br J Dermatol. 1994 October; 131(4):472-6. Additional antibodies can be generated using methods known in the art, e.g., as described herein, and screened and/or modified to produce antibodies with the desired specificity or affinity.

### Inflammatory Cytokines

The compositions described herein include a cytokine-binding domain that binds to and inhibits the activity of the cytokine. The binding domain is preferably specific for a selected inflammatory cytokine, e.g., tumor necrosis factor alpha (TNFα), Interleukin 17A (IL-17A), IL-12, IL-23, IL-6, IL-4 or Interferon gamma (IFNγ), and does not show substantial binding to other proteins or other cytokines. An inflammatory cytokine is one identified in an animal model or human studies as being associated with or causing activation of the innate and/or adaptive immune systems and contributing to the pathobiology of autoimmune and inflammatory conditions. Cytokine-binding domains useful in the present methods include those that bind to and sequesters the cytokine, preventing it from (or reducing) binding to and initiating inflammatory signaling in immune and non-immune cells, including production of other inflammatory cytokines by immune cells stimulated with one of the inflammatory cytokines. In some embodiments, the constructs include a plurality of cytokine-binding domains, which may all be of the same type, or may bind more than one cytokine.

Tumor Necrosis Factor Alpha (TNFα)

TNFα is a proinflammatory cytokine produced mainly by macrophages that has been implicated in autoimmune disease and cancer. A reference sequence of human TNFα is in GenBank at Accession No. NM_000594.3 (nucleic acid) and NP_000585.2 (protein). An exemplary protein sequence for human TNFα is as follows:

| (SEQ ID NO: 2) | |
|---|---|
| 1 | mstesmirdv elaeealpkk tggpqgsrrc lflslfsfli vagattlfcl lhfgvigpqr |
| 61 | eefprdlsli splaqavrss srtpsdkpva hvvanpqaeg qlqwlnrran allangvelr |
| 121 | dnqlvvpseg lyliysqvlf kgqgcpsthv llthtisria vsyqtkvnll saikspcgre |
| 181 | tpegaeakpw yepiylggvf qlekgdrlsa einrpdyldf aesgqvyfgi ial |

TNFα binding domains can include antibodies or antigen binding portions thereof or non-antibody binding domains as described herein, and/or can include natural ligands or TNFα-binding portions thereof, that inhibit binding of TNFα to its receptor and reduce or prevent the triggering of TNFα dependent inflammatory pathways in immune and non-immune cell types, including but not limited to inhibiting the maturation of dendritic cells, the expression of E-selectin and adhesion receptors in endothelial cells, the recruitment of immune cells into tissues and the enhancement of inflammatory mediators by T cells and keratinocytes. A number of TNFα-binding proteins are known in the art. For example, the TNF-α inhibitor etanercept is a recombinant form of a TNFα-binding portion of the TNF receptor 2 fused to an Fc domain (see U.S. Pat. No. 5,447,851). Infliximab is a human-mouse chimeric monoclonal anti-TNF antibody. Adalimumab is a fully human monoclonal antibody against TNF-α. Golimumab is a fully human IgG1 human TNF-α monoclonal antibody, and certolizumab is a Fab′ humanized fragment of an anti-TNF antibody attached to a polyethylene glycol moiety (PEGylated). See also Skurkovich et al., Journal of Immune Based Therapies, Vaccines and Antimicrobials, 2015, 4:1-8. The present constructs can include an entire antibody or receptor, or only a TNFα-binding portion thereof (e.g., the extracellular domain of the TNFα-R).

Interleukin 17A (IL-17A)

IL-17A is a proinflammatory cytokine produced mainly by activated T cells that has been implicated in several chronic inflammatory diseases including rheumatoid arthritis, psoriasis and multiple sclerosis. A reference sequence of human IL-17A precursor is in GenBank at Accession No. NM_002190.2 (nucleic acid) and NP_002181.1 (protein). An exemplary protein sequence for human IL-17A precursor is as follows:

| (SEQ ID NO: 3) | |
|---|---|
| 1 | mtpgktslvs lllllsleai vkagitiprn pgcpnsedkn fprtvmvnln ihnrntntnp |
| 61 | krssdyynrs tspwnlhrne dperypsviw eakcrhlgci nadgnvdyhm nsvpiqqeil |
| 121 | vlrrepphcp nsfrlekilv svgctcvtpi vhhva |

As amino acids 1-23 appear to be a signal sequence, the mature IL-17A is amino acids 24-155 of SEQ ID NO:3. IL-17A binding domains can include antibodies or antigen binding portions thereof as described herein, and/or can include natural ligands or IL-17A-binding portions thereof, that inhibit binding of IL-17A to its receptor and reduce or prevent the activation of inflammatory pathways in and the production of additional inflammatory mediators by keratinocytes, T cells and antigen presenting cells. A number of IL-17A-binding proteins are known in the art. For example, Dallenbach et al., Eur J Immunol. 2015 April; 45(4):1238-47, describe high-affinity neutralizing anti-IL-17 antibodies. Gerhardt et al., J. Mol. Biol. (2009) 394, 905-921, describe the neutralizing anti-IL-17 antibody CAT-2200. Ixekizumab (LY2439821) is a humanized IgG4 mAb that neutralizes IL-17. Secukinumab (AIN457) is a fully human mAb that neutralizes IL-17A. Brodalumab Others are known in the art; see, e.g., Lubberts et al., Arthritis Rheum. 2004; 50(2): 650-659; Cheng et al., Atherosclerosis. 2011; 215(2): 471-474; and WO2010102251. The present constructs can include an entire antibody or receptor, or only a IL-17A-binding portion thereof (e.g., the extracellular domain of the IL-17AR).

Interleukin 23 (IL-23A)

IL-23 is a heterodimer composed of the IL-23 alpha subunit protein (also referred to as p19) and the p40 subunit of interleukin 12 (IL12B, see below). A reference sequence of human IL-23A precursor is in GenBank at Accession No. NM_016584.2 (nucleic acid) and NP_057668.1 (protein). An exemplary protein sequence for human IL-23A precursor is as follows:

(SEQ ID NO: 4)

| | |
|---|---|
| 1 | mlgsravmll lllpwtaqgr avpggsspaw tqcqqlsqkl ctlawsahpl vghmdlreeg |
| 61 | deettndvph iqcgdgcdpq glrdnsqfcl grihqglify ekllgsdift gepsllpdsp |
| 121 | vgqlhasllg lsqllqpegh hwetqqipsl spsqpwqrll lrfkilrslq afvavaarvf |
| 181 | ahgaatlsp |

As amino acids 1-19 appear to be a signal sequence, the mature IL-23A is amino acids 20-189 of SEQ ID NO:4. IL-23 binding domains can include antibodies or antigen binding portions thereof as described herein that bind to IL-23A, and/or can include natural ligands or IL-23A-binding portions thereof, and that inhibit binding of IL-23A to its receptor and reduce or prevent preferential differentiation activation and cytokine production by IL-17 producing T cells. A number of IL-23A-binding proteins are known in the art. For example, U.S. Pat. Nos. 7,790,862 and 7,282,204 disclose anti-p19 antibodies. The extracellular part (fragment Gly24-Asn350) of the human IL-23 receptor (IL-23R, GenBank: AF461422.1) can also be used (see Kuchar̆ et al., Proteins. 2014 June; 82(6): 975-989), as can the proteins encoded by IL-23Ra (HuIL23Ra)-chain mRNA transcripts that lack exon 9, which are also known as HuIL23RaΔ9 or “Δ9”), see Yu and Gallagher, Journal of Immunology, 2010, 185: 7302-7308, and the ABD-derived p19-targeted variants, called ILP binders (e.g., ILP030, ILP317 and ILP323), described in Kr̆íz̆ová et al., Autoimmunity. 2017 March; 50(2):102-113. A number of therapeutic antibodies are known in the art as well, including Ustekinumab (also known as CNTO 1275), Tildrakizumab, also known as MK-3222 or SCH900222, which is a human immunoglobulin G1 (IgG1); Guselkumab, or CNTO 1959, which is a humanized IgG1 monoclonal antibody; BI655066, a human IgG1 monoclonal antibody; and MP-196, another monoclonal antibody targeting IL-23. See, e.g., Kollipara et al., Skin Therapy Letter. 2015; 20(2). See also U.S. Pat. No. 8,563,697 and Clarke et al., MAbs. 2010 September-October; 2(5): 539-549 (h6F6 human mAb). Monomeric IL-12p80, or an inactive but p19-binding fragment thereof, can also be used. The present constructs can include an entire antibody or receptor, or only an IL-23-binding portion thereof (e.g., the extracellular domain of the IL-23R).

Interleukin 6 (IL-6)

Interleukin 6 is primarily produced at sites of acute and chronic inflammation, where it is secreted into the serum and induces an inflammatory response through the IL-6 receptor, alpha. There are multiple splice variants. A reference sequence of human IL-6 isoform 1 precursor is in GenBank at Accession No. NM_000600.4 (nucleic acid) and NP_000591.1 (protein). A reference sequence of human IL-6 isoform 2 precursor is in GenBank at Accession No. NM_001318095.1 (nucleic acid) and NP_001305024.1 (protein). An exemplary protein sequence for human IL-6 isoform 1 is as follows:

| (SEQ ID NO: 5) | |
|---|---|
| 1 | mnsfstsafg pvafslglll vlpaafpapv ppgedskdva aphrqpltss eridkqiryi |
| 61 | ldgisalrke tcnksnmces skealaennl nlpkmaekdg cfqsgfneet clvkiitgll |
| 121 | efevyleylq nrfesseeqa ravqmstkvl iqflqkkakn ldaittpdpt tnaslltklq |
| 181 | aqnqwlgdmt thlilrsfke flqsslralr qm |

As amino acids 1-29 appear to be a signal sequence, the mature IL-6 isoform 1 protein can be considered amino acids 30-212 or 33-212.

An exemplary protein sequence for human IL-6 isoform 2 (the shorter isoform, with a truncated N-terminus) is as follows:

| (SEQ ID NO: 6) | |
|---|---|
| 1 | mcesskeala ennlnlpkma ekdgcfqsgf neetclvkii tgllefevyl eylqnrfess |
| 61 | eeqaravqms tkvliqflqk kaknldaitt pdpttnasll tklqaqnqwl qdmtthlilr |
| 121 | sfkeflqssl ralrqm |

IL-6 binding domains can include antibodies or antigen binding portions thereof as described herein that bind to IL-6, and/or can include natural ligands or IL-6-binding portions thereof, that inhibit binding of IL-6 to its receptor and reduce or prevent enhanced production of other inflammatory cytokines (IL-13, IL-17A, IL-4, IL-21) by CD4+ T cells. A number of IL-6-binding proteins are known in the art. For example, Siltuximab (also known as CNTO 328) is a human/mouse chimeric anti-IL-6 monoclonal antibody. Clazakizumab (also known as ALD518 and BMS-945429) is an aglycosylated, humanized anti-IL-6 monoclonal antibody. Sirukumab (also known as CNTO-136) is a human monoclonal antibody. Olokizumab (humanized), elsilimomab (mouse mAb, also known as B-E8) are other anti-IL-6 antibodies. The present constructs can include an entire antibody or receptor, or only an IL-6-binding portion thereof (e.g., the extracellular domain of the IL-6-R).

Interleukin 4 (IL-4)

Interleukin 4 is a cytokine produced by activated T cells that mediates important pro-inflammatory functions in asthma.

There are multiple splice variants. A reference sequence of human IL-4 isoform 1 precursor is in GenBank at Accession No. NM_000589.3 (nucleic acid) and NP_000580.1 (protein). A reference sequence of human IL-4 isoform 2 precursor is in GenBank at Accession No. NM_172348.2 (nucleic acid) and NP_758858.1 (protein). An exemplary protein sequence for human IL-4 isoform 1 is as follows:

| (SEQ ID NO: 7) | |
|---|---|
| 1 | mgltsqllpp lffllacagn fvhghkcdit lqeiiktlns lteqkticte ltvtdifaas |
| 61 | kntteketfc raatvlrqfy shhekdtrcl gataqqfhrh kqlirflkrl drnlwglagl |
| 121 | nscpvkeanq stlenflerl ktimrekysk css |

As amino acids 1-24 appear to be a signal sequence, the mature IL-4 isoform 1 protein can be considered amino acids 25-153 or 28-149.

An exemplary protein sequence for human IL-4 isoform 2 is as follows:

| (SEQ ID NO: 8) | |
|---|---|
| 1 | mgltsqllpp lffllacagn fvhghkcdit lqeiiktlns lteqknttek etfcraatvl |
| 61 | rqfyshhekd trclgataqq fhrhkqlirf lkrldrnlwg laglnscpvk eanqstlenf |
| 121 | lerlktimre kyskcss |

As amino acids 1-24 appear to be a signal sequence, the mature IL-4 isoform 1 protein can be considered amino acids 25-137 or 28-133.

IL-4 binding domains can include antibodies or antigen binding portions thereof as described herein that bind to IL-4, and/or can include natural ligands or IL-4-binding portions thereof, that inhibit binding of IL-4 to its receptor and reduce or prevent the stimulation of activated B cell and T cell proliferation, the differentiation of B cells into plasma cells and B cell class switching to IGE production. A number of IL-4-binding proteins are known in the art. For example, Pascolizumab (SB 240683) is a humanized mAb that binds to and blocks IL-4. All or part of the soluble recombinant human IL-4 receptor can also be used, e.g., altrakincept. The present constructs can include an entire antibody or receptor, or only an IL-4-binding portion thereof (e.g., the extracellular domain of the IL-4R).

Interleukin 12 (IL-12)

IL-12 is a disulfide-linked heterodimer composed of the 35-kD IL-12A subunit (also known as Interleukin-12 alpha subunit) and the 40-kD IL-12B subunit (also known as Interleukin-12 beta subunit and p40 subunit). IL-12 is expressed by activated macrophages and is important for development and maintenance of Th2 and Th1 cells. IL-12 has been associated with MS and both atopic and non-atopic asthma in children.

A reference sequence of human IL-12A precursor is in GenBank at Accession No. NM_000882.3 (nucleic acid) and NP_000873.2 (protein). An exemplary protein sequence for human IL-12A precursor is as follows:

| (SEQ ID NO: 9) | |
|---|---|
| 1 | mwppgsasqp ppspaaatgl hpaarpvslq crlsmcpars lllvativll dhlslarnlp |
| 61 | vatpdpgmfp clhhsqnllr aysnmlqkar qtlefypcts eeidheditk dktstveacl |
| 121 | pleltknesc lnsretsfit ngsclasrkt sfmmalclss iyedlkmyqv efktmnakll |
| 181 | mdpkrqifld qnmlavidel mqalnfnset vpqkssleep dfyktkiklc illhafrira |
| 241 | vtidrvmsyl nas |

The mature IL-12A is amino acids 51-253 or 57-253.

A reference sequence of human IL 12B precursor is in GenBank at Accession No. NM_002187.2 (nucleic acid) and NP_002178.2 (protein). An exemplary protein sequence for human IL-12A precursor is as follows:

| (SEQ ID NO: 10) | |
|---|---|
| 1 | mchqqlvisw fslvflaspl vaiwelkkdv yvveldwypd apgemvvltc dtpeedgitw |
| 61 | tldqssevlg sgktltiqvk efgdagqytc hkggevlshs llllhkkedg iwstdilkdq |
| 121 | kepknktflr ceaknysgrf tcwwlttist dltfsvkssr gssdpqgvtc gaatlsaery |
| 181 | rgdnkeyeys vecqedsacp aaeeslpiev mvdavhklky enytssffir diikpdppkn |
| 241 | lqlkplknsr qvevsweypd twstphsyfs ltfcvqvqgk skrekkdrvf tdktsatvic |
| 301 | rknasisvra qdryysssws ewasvpcs |

As amino acids 1-22 appear to be a signal sequence, the mature IL-12B is amino acids 23-328.

IL-12 binding domains can include antibodies or antigen binding portions thereof as described herein that bind to IL-12A and/or IL-12B, and/or can include natural ligands or IL-12-binding portions thereof, that inhibit binding of IL-12 to its receptor and reduce or prevent the differentiation of naïve T cells into Th1 cells, the stimulation of interferon gamma and TNFalpha production by T cells and natural killer (NK) cells. A number of IL-12-binding proteins are known in the art. For example, anti-IL-12 antibodies are described in WO2006069036; WO2002012500; U.S. Pat. Nos. 8,404,819; 8,563,697; Krueger et al., N Engl J Med 2007; 356:580-592; and Clarke et al., MAbs. 2010 September-October; 2(5): 539-549 (h6F6 human mAb). A number of therapeutic antibodies are known in the art as well, including ustekinumab (a human monoclonal antibody also known as CNTO 1275) and briakinumab (a fully human monoclonal antibody also known as ABT-874); both bind to IL-12B and thus targets interleukin-12 (IL-12) and interleukin-23 (IL-23). They have been in clinical trials for plaque psoriasis, psoriatic arthritis, and multiple sclerosis, rheumatoid arthritis, inflammatory bowel diseases and Crohn's disease. Monomeric IL-12p40, or an inactive but IL-12A-binding fragment thereof, can also be used. The present constructs can include an entire antibody or receptor, or only a IL-12-binding portion thereof (e.g., the extracellular domain of the IL-12R).

Interferon Gamma (IFNγ)

IFNγ is a soluble cytokine secreted by immune cells in response to infection. The active protein is a homodimer.

A reference sequence of human IFNγ precursor is in GenBank at Accession No. NM_000619.2 (nucleic acid) and NP_000610.2 (protein). An exemplary protein sequence for human IFNγ precursor is as follows:

```
(SEQ ID NO: 11)
    1 mkytsyilaf qlcivlgslg cycqdpyvke aenlkkyfna ghsdvadngt lflgilknwk
  61 eesdrkimqs qivsfyfklf knfkddqsiq ksvetikedm nvkffnsnkk krddfekltn
121 ysvtdlnvqr kaiheliqvm aelspaaktg krkrsqmlfr grrasq
```

The mature form of IFNγ is likely to be amino acids 24-161 of SEQ ID NO:11.

IFNγ binding domains can include antibodies or antigen binding portions thereof or non-antibody binding domains as described herein, and/or can include natural ligands or IFNγ-binding portions thereof, that inhibit binding of IFNγ to its receptor and reduce or prevent the upregulation of MHC II molecule expression and activation immune interferon response pathways in immune cell types (macrophages, dendritic cells, T cells) and non-immune cell types. A number of IFNγ-binding proteins are known in the art. For example, Fontolizumab is a humanized monoclonal antibody developed for use in RA and Crohn's disease. See also Skurkovich et al., Journal of Immune Based Therapies, Vaccines and Antimicrobials, 2015, 4:1-8.

Exemplary Sequences

A set of exemplary sequence of cytokine binding domains follows.

### Etanercept

(SEQ ID NO: 12)
LPAQVAFTPYAPEPGSTCRLREYYDQTAQMCCSKCSPGQHAKVFCTKT
SDTVCDSCEDSTYTQLWNWVPECLSCGSRCSSDQVETQACTREQNRIC
TCRPGWYCALSKQEGCRLCAPLRKCRPGFGVARPGTETSDVVCKPCAP
GTFSNTTSSTDICRPHQICNVVAIPGNASMDAVCTSTSPTRSMAPGAV
HLPQPVSTRSQHTQPTPEPSTAPSTSFLLPMGPSPPAEGSTGDEPKSC
DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVS
LTCLVKGFYPSDIAVEWESNGQPENNYKTTPPVLDSDGSFFLYSKLTV
DKSRWQQGNVFSCSVMHEALHNHYTQKSLSLSPGK
(U.S. Pat. No. US07276477)

### Infliximab

Infliximab heavy chain variable region:
(SEQ ID NO: 13)
EVKLEESGGGLVQPGGSMKLSCVASGFIFSNHWMNWVRQSPEKGLEWV
AEIRSKSINSATHYAESVKGRFTISRDDSKSAVYLQMTDLRTEDTGVY
YCSRNYYGSTYDYWGQGTTLTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKT
(Liang et al. (2013) J. Biol.
Chem. 288: 13799-13807)
Infliximab light chain variable region:
(SEQ ID NO: 14)
DILLTQSPAILSVSPGERVSFSCRASQFVGSSIHWYQQRTNGSPRLLI
KYASESMSGIPSRFSGSGSGTDFTLSINTVESEDIADYYCQQSHSWPF
TFGSGTNLEVKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
(Liang et al. (2013) J. Biol.
Chem. 288: 13799-13807)

### Adalimumab

Adalimumab heavy chain variable region:
(SEQ ID NO: 15)
EVQLVESGGGLVQPGRSLRLSCAASGFTFDDYAMHWVRQAPGKGLEWV
SAITWNSGHIDYADSVEGRFTISRDNAKNSLYLDMNSLRAEDTAVYYC
AKVSYLSTASSLDYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKI
(Hu et al. (2013) J. Biol. Chem. 288: 27059-27067)
Adalimumab light chain variable region:
(SEQ ID NO: 16)
DIQMTQSPSSLSASVGDRVTITCRASQGIRNYLAWYQQKPGKAPKLLI
YAASTLQSGVPSRFSGSGSGTDFTLTISSLQPEDVATYYCQRYNRAPY
TFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGE
(Hu et al. (2013) J. Biol. Chem. 288: 27059-27067)

### Golimumab

Golimumab heavy chain variable region:
(SEQ ID NO: 17)
QVQLVESGGGVVQPGRSLRLSCAASGFIFSSYAMHWVRQAPGNGLEWV
AFMSYDGSNKKYADSVKGRFTISRDNSKNTLYLQMNSLRAEDTAVYYC
ARDRGIAAGGNYYYYGMDVWGQGTTVTVSS
(U.S. Patent 2015/0166649)
Golimumab light chain variable region:
(SEQ ID NO: 18)
EIVLTQSPATLSLSPGERATLSCRASQSVYSYLAWYQQKPGQAPRLLI
YDASNRATGIPARFSGSGSGTDFTLTISSLEPEDFAVYYCQQRSNWPP
FTFGPGTKVDIK
(U.S. Patent 2015/0166649)

### Certolizumab

Certolizumab heavy chain variable region:
(SEQ ID NO: 19)
EVQLVESGGGLVQPGGSLRLSCAASGYVFTDYGMNWVRQAPGKGLEWM
GWINTYIGEPIYADSVKGRFTFSLDTSKSTAYLQMNSLRAEDTAVYYC
ARGYRSYAMDYWGQGTLVTVSS
(U.S. Patent US2015/0166649)
Certolizumab light chain variable region:
(SEQ ID NO: 20)
DIQMTQSPSSLSASVGDRVTITCKASQNVGTNVAWYQQKPGKAPKALI
YSASFLYSGVPYRFSGSGSGTDFTLTISSLQPEDFATYYCQQYNIYPL
TFGQGTKVEIK
(U.S. Patent US2015/0166649)

### Ixekizumab

Ixekizumab heavy chain:
(SEQ ID NO: 21)
QVQLVQSGAEVKKPGSSVKVSCKASGYSFTDYHIHWVRQAPGQGLEWM
GVINPMYGTTDYNQRFKGRVTITADESTSTAYMELSSLRSEDTAVYYC
ARYDYFTGTGVYWGQGTLVTVSSASTKGPSVFPLAPCSRSTSESTAAL
GCLVKDYFPEPVTVSWNSGALTSGVHTFPAVLQSSGLYSLSSVVTVPS
SSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNG
QPENNYKTTPPVLDSDGSFFLYSRLTVDKSRWQEGNVFSCSVMHEALH
NHYTQKSLSLSLG
(see drugbank.ca/drugs/DB11569)
Ixekizumab light chain:
(SEQ ID NO: 22)
DIVMTQTPLSLSVTPGQPASISCRSSRSLVHSRGNTYLHWYLQKPGQS
PQLLIYKVSNRF
IGVPDRFSGSGSGTDFTLKISRVEAEDVGVYYCSQSTHLPFTFGQGTK
LEIKRTVAAPSV
FIFPPSDEQLKSGTASVVCLLNNFYPREAKVQWKVDNALQSGNSQESV
TEQDSKDSTYSLSSTLTLSKADYEKHKVYACEVTHQGLSSPVTKSFNR
GEC
(https://www.drugbank.ca/drugs/DB11569)

### Secukinumab (AIN457)

Secukinumab heavy chain variable region:
(SEQ ID NO: 23)
EVQLVESGGGLVQPGGSLRLSCAASGFTFSNWMNWVRQAPGKGLEWVA
AINQDGSEKYYVGSVKGRFTISRDNAKNSLYLQMNSLRVEDTAVYYCV
RDYYDILTDYYIHWYFDLWGRGTLVTVSS
(U.S. Patent US2013/0202610)
Secukinumab light chain variable region:
(SEQ ID NO: 24)
EIVLTQSPGTLSLSPGERATLSCRASQSVSSSYLAWYQQKPGQAPRLL
IYGASSRATGIPDRFSGSGSGTDFTLTISRLEPEDFAVYYCQQYGSSP
CTFGQGTRLEIKR
(U.S. Patent US2013/0202610)

### Ustekinumab (Also Known as CNTO 1275)

Ustekinumab heavy chain:
(SEQ ID NO: 25)
EVQLVQSGAEVKKPGESLKISCKGSGYSFTTYWLGWVRQMPGKGLDWI
GIMSPVDSDIRYSPSFQGQVTMSVDKSITTAYLQWNSLKASDTAMYYC
ARRRPGQGYFDFWGQGTLVTVSSSSTKGPSVFPLAPSSKSTSGGTAAL
GCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGP
SVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHE
ALHNHYTQKSLSLSPGK
(WO/2014/004436)
Ustekinumab light chain:
(SEQ ID NO: 26)
DIQMTQSPSSLSASVGDRVTITCRASQGISSWLAWYQQKPEKAPKSLI
YAASSLQSGVPSRFSGSGSGTDFTLTISSLQPEDFATYYCQQYNIYPY
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
(WO/2014/004436)

### Guselkumab, (Also Known as CNTO 1959)

Guselkumab heavy chain:
(SEQ ID NO: 27)
EVQLVQSGAEVKKPGESLKISCKGSGYSFSNYWIGWVRQMPGKGLEWM
GIIDPSNSYTRYSPSFQGQVTISADKSISTAYLQWSSLKASDTAMYYC
ARWYYKPFDVWGQGTLVTVSSASTKGPSVFPLAPSSKSTSGGTAALGC
LVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEAL
HNHYTQKSLSLSPG
(WO/2014/004436)
Guselkumab light chain:
(SEQ ID NO: 28)
QSVLTQPPSVSGAPGQRVTISCTGSSSNIGSGYDVHWYQQLPGTAPKW
YGNSKRPSGV
PDRFSGSKSGTSASLAITGLQSEDEADYYCASWTDGLSLVVFGGGTKL
TVLGQPKAAPSVTLFPPSSEELQANKATLVCLISDFYPGAVTVAWKAD
SSPVKAGVETTTPSKQSNNKYAASSYLSLTPEQWKSHRSYSCQVTHEG
STVEKTVAPTECS
(WO/2014/004436)

### Siltuximab (Also Known as CNTO 328)

Siltuximab heavy chain:
(SEQ ID NO: 29)
EVQLVESGGKLLKPGGSLKLSCAASGFTFSSFAMSWFRQSPEKRLEWV
AEISSGGSYTYYPDTVTGRFTISRDNAKNTLYLEMSSLRSEDTAMYYC
ARGLWGYYALDYWGQGTSVTVSSASTKGPSVFPLAPSSKSTSGGTAAL
GCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEK
TISKAKGQPREPQVYTLPPSRDELTKNQVSL
TCLVKGFYPSDIAVEWESNGQPENNYKTTPPVLDSDGSFFLYSKLTVD
KSRWQQGNVFSCSVMHEALHNHYTQKSLSLSPGK
(drugbank.ca/drugs/DB09036)
Siltuximab light chain:
(SEQ ID NO: 30)
QIVLIQSPAIMSASPGEKVTMTCSASSSVSYMYWYQQKPGSSPRLLIY
DTSNLASGVPVR
FSGSGSGTSYSLTISRMEAEDAATYYCQQWSGYPYTFGGGTKLEIKRT
VAAPSVFIFPPS
DEQLKSGTASVVCLLNNFYPREAKVQWKVDNALQSGNSQESVTEQDSK
DSTYSLSSTLTLSKADYEKHKVYACEVTHQGLSSPVTKSFNRGEC
(drugbank.ca/drugs/DB09036)

### Clazakizumab (Also Known as ALD518 and BMS-945429)

Clazakizumab heavy chain variable region:
(SEQ ID NO: 31)
IQMTQSPSSLSASVGDRVTITCQASQSINNELSWYQQKPGKAPKLLIY
RASTLASGVPSR
FSGSGSGTDFTLTISSLQPDDFATYYCQQGYSLRNIDNA
(U.S Patent 2016/0130340)
Clazakizumab light chain variable region:
(SEQ ID NO: 32)
EVQLVESGGGLVQPGGSLRLSCAASGFSLSNYYVTWVRQAPGKGLEWV
GIIYGSDETAYATSAIGRFTISRDNSKNTLYLQMNSLRAEDTAVYYCA
RDDSSDWDAKFNL
(U.S Patent 2016/0130340)

### Olokizumab

Olokizumab heavy chain variable region:
(SEQ ID NO: 33)
EVQLVESGGGLVQPGGSLRLSCAASGFNFNDYFMNWVRQAPGKGLEWV
AQMRNKNYQYGTYYAESLEGRFTISRDDSKNSLYLQMNSLKTEDTAVY
YCARESYYGFTSYWGQGTLVTVSSASTKGPSVFPLAPCSRSTSESTAA
LGCLVKDYFPEPVTVSWNSGALTSGVHTFPAVLQSSGLYSLSSVVTVP
SSSLGTKTYTCNVDHKPSNTKVDKRVES
(Shaw et al. (2014) MAbs 6: 773-781)
Olokizumab light chain variable region:
(SEQ ID NO: 34)
DIQMTQSPSSLSASVGDRVTITCQASQDIGISLSWYQQKPGKAPKLLI
YNANNLADGVPSRFSGSGSGTDFTLTISSLQPEDFATYYCLQHNSAPY
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
(Shaw et al. (2014) MAbs 6: 773-781)

### Pascolizumab (Also Known as SB 240683)

Pascolizumab heavy chain:
(SEQ ID NO: 35)
QVTLRESGPALVKPTQTLTLTCTFSGFSLSTSGMGVSWIRQPPGKGLE
WLAHIYWDDDKRYNPSLKSRLTISKDTSRNQVVLTMTNMDPVDTATYY
CARRETVFYWYFDVWGRGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEV
HNAKTKRVVSVLPREEQYNSTYTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVM
HEALHNHYTQKSLSLSPGK
(WO 2009/068649)
Pascolizumab light chain:
(SEQ ID NO: 36)
DIVLTDRVTIQSPSSLSASVGTCKASQSVDYDGDSYMNWYQQKPGKAP
KLLIYAASNLESGIPSRFSGSGSGTDFTFTISSLQPEEDPPTDFIATY
GQGTYCQQSNKVEIKRTVAAPSVFIFPPSDEQLKSGTASVVCLLNNFY
PREAKVQWKVDNALQSGNSQEYSLSSHKVYASVTEQDSKDSTTLTLCE
VTSKADYEKHQGLSSPVTKSFNRGEC
(WO 2009/068649)

### Briakinumab (Also Known as ABT-874)

Briakinumab heavy chain:
(SEQ ID NO: 37)
QVQLVESGGGVVQPGRSLRLSCAASGFTFSSYGMHWVRQAPGKGLEWV
AFIRYDGSNKYYADSVKGRFTISRDNSKNTLYLQMNSLRAEDTAVYYC
KTHGSHDNWGQGTMVTVSSASTKGPSVFPLAPSSKSTSGGTAALGCLV
KDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHN
HYTQKSLSLSPGK
(WO/2014/004436)
Briakinumab light chain:
(SEQ ID NO: 38)
QSVLTQPPSVSGAPGQRVTISCSGSRSNIGSNTVKWYQQLPGTAPKLL
IYYNDQRPSGVPDRFSGSKSGTSASLAITGLQAEDEADYYCQSYDRYT
HPALLFGTGTKVTVLGQPKAAPSVTLFPPSSEELQANKATLVCLISDF
YPGAVTVAWKADSSPVKAGVETTTPSKQSNNKYAASSYLSLTPEQWKS
HRSYSCQVTHEGSTVEKTVAPTECS
(WO/2014/004436)

### Linkers and Conjugation

As noted above, the collagen-targeted constructs described herein can be, e.g., fusion proteins that are encoded by a single nucleic acid, or they can be made by conjugating two separate proteins together. When the constructs are fusion proteins, the two sequences can be immediately connected or connected via a peptide linker, e.g., a gly-ser linker or an immunoglobulin hinge region or portion thereof.

Examples of suitable linkers include (GGGGS)n, (SEQ ID NO:39), the Fc interlinker from human IgG1 Cm residues 297-322: NSTYRVVSVLTVLHQDWLNGKEYKCK (SEQ ID NO:40), and the HAS interlinker from the D3 domain of human serum albumin: FQNALLVRYTKKVPQVSTPTLVEVS (SEQ ID NO:41). See Fang et al., Chines. Sci. Bull, 2003, 48: 1912-1918, incorporated by reference in its entirety. In some embodiments, the linker is GGGGS) (SEQ ID NO:42); (GGGGS)₂ (SEQ ID NO:43); (GGGGS)₃ (SEQ ID NO:44); (GGGGS)₄ (SEQ ID NO:45); (GGGGS)₅ (SEQ ID NO:46); or (GGGGS)₆ (SEQ ID NO:47). Other linkers are provided, for example, in U.S. Pat. No. 5,525,491; Alfthan et al, Protein Eng., 1995, 8:725-731; Shan et al, J Immunol., 1999, 162:6589-6595; Newton et al, Biochemistry, 1996, 35:545-553; Megeed et al.; Biomacromolecules, 2006, 7:999-1004; and Perisic et al., Structure, 1994, 12: 1217-1226; each of which is incorporated by reference in its entirety.

In some embodiments, the polypeptide linkers are encoded by a polynucleotide that also encodes two or more domains linked by the polypeptide linker (e.g., a fusion protein). Such polynucleotides can be produced by assembling or synthesizing a polynucleotide encoding a first domain, a first polypeptide linker, and a second domain. In some embodiments, the polynucleotide can further encode a second polypeptide linker and a third domain. The polynucleotide can then be expressed, according to the methods provided herein and known in the art, to produce a fusion protein comprising two or more domains connected by the linker.

In some embodiments, the domains are expressed separately and the polypeptide linker is used to attach two or more domains to each other after expression. In such embodiments, a first domain is contacted with a first polypeptide linker under conditions suitable for the formation of a chemical bond between the first domain and the first polypeptide linker. A second domain is then contacted with the conjugate formed by the first domain and the first polypeptide linker, under conditions suitable for the formation of a chemical bond between the first polypeptide linker and the second domain. Additional domains can be conjugated to the first and/or second domains, or to the first linker, by utilizing similar techniques. Conditions suitable for the formation of chemical bonds between polypeptide linkers and domains are provided, for example, in Hermanson, Bioconjugate Techniques, 2013, 3d ed., Academic Press, London, UK, Waltham Mass., and San Diego, Calif., which is incorporated by reference in its entirety.

In some embodiments, the domains are covalently associated by a chemical coupling. Any suitable chemical linker can be used to covalently associate the domains provided herein. Chemical coupling of antibodies to each other is described, for example, in Wong et al., Scand. J. Rheumatol, 2000, 29:282-287; Jung et al., Eur. J. Immunol, 1991, 21:2431-2435; Tutt et al, J. Immunol, 1991, 147:60-69; French, Methods Mol. Biol, 1998, 80: 121-134; and Gavrilyuk et al., Bioorg. Med. Chem. Lett, 2009, 19:3716-3720; each of which is incorporated by reference in its entirety. In such embodiments, a first domain is contacted with a first chemical coupling reagent under conditions suitable for the formation of a chemical bond between the first domain and the first chemical coupling reagent. A second domain is then contacted with the conjugate formed by the first domain and the first chemical coupling reagent, under conditions suitable for the formation of a chemical bond between the first chemical coupling reagent and the second domain. Additional domains can be conjugated to the first and/or second domains, or to the first chemical coupling reagent, by utilizing similar techniques. Conditions suitable for the formation of chemical bonds between chemical coupling reagents and domains are provided, for example, in Hermanson, Bioconjugate Techniques, 2013, 3d ed., Academic Press, London, UK, Waltham Mass., and San Diego, Calif., which is incorporated by reference in its entirety.

Any suitable coupling reagent can be used when chemically coupling MIACs. Coupling reagents include zero-length crosslinkers, homobifunctional crosslinkers, heterobifunctional crosslinkers, trifunctional crosslinkers, dendrimers and dendrons, chemoselective and bioorthogonal reagents, and the like. Illustrative suitable coupling reagents include, for example, m-maleimidobenzoic acid, N-hydroxysuccinimide ester, glutaraldehyde, and carbodiimides. Other suitable reagents for chemical coupling, and methods of their use, are described, for example, in Hermanson, Bioconjugate Techniques, 2013, 3d ed., Academic Press, London, UK, Waltham Mass., and San Diego, Calif., which is incorporated by reference in its entirety. Chemical coupling of antibodies to each other is described, for example, in Wong et al., Scand. J. Rheumatol, 2000, 29:282-287; Jung et al., Eur. J. Immunol, 1991, 21:2431-2435; Tutt et al, J Immunol, 1991, 147:60-69; French, Methods Mol Biol, 1998, 80: 121-134; and Gavrilyuk et al., Bioorg. Med. Chem. Lett, 2009, 19:3716-3720; each of which is incorporated by reference in its entirety.

In some embodiments, the chemical coupling is via a spacer. In some embodiments, the spacer is a molecule selected from a polymer, a polypeptide, a carbohydrate (e.g., dextran), or the like. In particular embodiments, the spacer is a poly(ethylene) glycol (PEG) polymer. In some embodiments, the PEG has a molecular weight in the range of about 2.5 kDa to about 50 kDa. PEG reagents for chemical coupling, and methods of their use, are described, for example, in Hermanson, Bioconjugate Techniques, 2013, 3d ed., chapter 18, Academic Press, London, UK, Waltham Mass., and San Diego, Calif., which is incorporated by reference in its entirety.

In some embodiments, the domains are non-covalently associated with each other. The non-covalent association can be any suitable covalent linkage.

In some embodiments, the non-covalent association is in the form of a specific interaction between two molecules. For example, in some embodiments, the non-covalent association is an interaction between avidin and biotin. In some embodiments, the avidin is selected from a streptavidin and a neutravidin. In these embodiments, an avidin molecule is attached to one domain and a biotin molecule is attached to another domain. The domains then associate as a result of the specific, high affinity interaction between the avidin and the biotin. Avidin-biotin systems, and methods of their use, are described, for example, in Hermanson, Bioconjugate Techniques, 2013, 3d ed., chapter 11, Academic Press, London, UK, Waltham Mass., and San Diego, Calif., which is incorporated by reference in its entirety.

See also WO2016115274, which is incorporated herein in its entirety.

### Nucleic Acids

In addition, where the collagen-targeted constructs are single (fusion) proteins, provided herein are nucleic acids that encode the collagen-targeted constructs, as well as vectors, preferably expression vectors, containing a nucleic acid encoding the collagen-targeted constructs described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses. In some embodiments, the collagen-targeted constructs are made and/or administered using nucleic acids.

A vector can include a nucleic acid encoding the collagen-targeted constructs described herein in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids that encode the collagen-targeted constructs described herein.

The recombinant expression vectors of the invention can be designed for expression of the collagen-targeted constructs in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified collagen-targeted constructs can be used as therapeutic agents in the methods described herein.

To maximize recombinant protein expression in E. coli is to express the protein in host bacteria with an impaired capacity to proteolytically cleave the recombinant protein (Gottesman, S., (1990) Gene Expression Technology: Methods in Enzymology 185, Academic Press, San Diego, Calif., 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

Also provided herein are host cells that include a nucleic acid molecule described herein, e.g., a nucleic acid molecule within a recombinant expression vector or a nucleic acid molecule containing sequences that allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a protein can be expressed in bacterial cells such as E. coli, insect cells, yeast or mammalian cells (such as Chinese hamster ovary cells (CHO) or COS cells). Other suitable host cells are known to those skilled in the art.

Following expression, the antibodies and fragments thereof can be isolated. An antibody or fragment thereof can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3rd edition,” Springer-Verlag, New York City, N.Y. The degree of purification necessary will vary depending on the desired use. In some instances, no purification of the expressed antibody or fragments thereof will be necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### Gene Therapy

Nucleic acids encoding a collagen-targeted construct can be incorporated into a gene construct to be used as a part of a gene therapy protocol. Thus described herein are expression vectors for in vivo transfection and expression of a polynucleotide that encodes a collagen-targeted construct, preferably targeted expression in particular cell types, especially epithelial cells. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include WCrip, WCre, W2 and WAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

In addition to viral transfer methods, such as those illustrated above, non-viral methods can also be employed to cause expression of a nucleic acid compound described herein (e.g., a nucleic acid encoding a collagen-targeted construct) in the tissue of a subject. Typically, non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

In some embodiments, a nucleic acid encoding a collagen-targeted construct described herein is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Methods of Treatment

The methods described herein include methods for the treatment of disorders associated with an inflammatory response in tissues that express Collagen VII, i.e., the lungs, skin, and gut. In some embodiments, the disorder is rheumatoid arthritis, psoriasis, inflammatory bowel disease, asthma, atopic dermatitis, dermatomyositis, systemic or cutaneous lupus erythematosus, scleroderma, graft-versus-host disease, drug hypersensitivity responses or organ transplant rejection (i.e., after transplant of an organ that includes an epithelial layer, e.g., skin, lung, or part of the gut). Generally, the methods include administering a therapeutically effective amount of a collagen VII-targeted construct as described herein, to a subject who is in need of, or who has been determined to be in need of, such treatment.

As used in this context, to “treat” means to ameliorate at least one symptom of the disorder associated with an inflammatory response in tissues that express Collagen VII. Administration of a therapeutically effective amount of a compound described herein for the treatment of a condition described herein will result in decreased inflammation.

In some embodiments, collagen-targeted constructs as described herein that include a TNFα-binding domain can be used, e.g., to treat rheumatoid arthritis, hidradenitis suppurativa, psoriasis, ankylosing spondylitis, ulcerative colitis and Crohn's disease. See, e.g., Ali et al., Drug Healthc Patient Saf. 2013; 5: 79-99. These constructs can also be used, e.g., to treat graft versus host disease, e.g., in the skin or gut; see, e.g., Park et al., Korean J Intern Med. 2014 September; 29(5): 630-636; Levine et al., Blood. 2008 Feb. 15; 111(4): 2470-2475; Choi et al., Biol Blood Marrow Transplant. 2012 October; 18(10):1525-32 and organ transplant rejection; see, e.g., Shen et al., J Am Soc Nephrol. 2009 20(5): 1032-1040.

In some embodiments, collagen-targeted constructs as described herein that include a IL-17A-binding domain can be used, e.g., to treat multiple sclerosis, rheumatoid arthritis, psoriasis, inflammatory bowel disease, ulcerative colitis, Crohn's disease and graft rejection. See, e.g., Kellner, Ther Adv Musculoskelet Dis. 2013 June; 5(3): 141-152 and Wang et al., Inflamm Bowel Dis. 2015 May; 21(5):973-84 and Antonysamy, et al., J Immunol. 1999. 162(1): 577-584.

In some embodiments, collagen-targeted constructs as described herein that include a IL-23A-binding domain can be used, e.g., to treat multiple sclerosis, rheumatoid arthritis, psoriasis, inflammatory bowel disease, ulcerative colitis and Crohn's disease. See, e.g., Kollipara et al., Skin Therapy Letter. 2015; 20(2); Guttman-Yassky et al., Expert Opin Biol Ther. 2013 April; 13(4): 10.1517/14712598.2013.758708. Collagen-targeted constructs as described herein that include a IL-23A-binding domain can also be used, e.g., to treat lupus, e.g., CLE, see, e.g., Presto et al., Lupus (2017 26:115-118.

In some embodiments, collagen-targeted constructs as described herein that include a IL-6-binding domain can be used, e.g., to treat rheumatoid arthritis (RA) or systemic onset juvenile idiopathic arthritis (soJIA) (Hennigan and Kavanaugh, Ther Clin Risk Manag. 2008 August; 4(4): 767-775) or graft rejection (Shen et al., J Am Soc Nephrol. 2009 20(5): 1032-1040).

In some embodiments, collagen-targeted constructs as described herein that include an IL-4-binding domain can be used, e.g., to treat asthma (Steinke et al., Immunol Allergy Clin North Am. 2004 November; 24(4):599-614, v; Kau and Korenblat, Curr Opin Allergy Clin Immunol. 2014 December; 14(6): 570-575) or atopic dermatitis (Guttman-Yassky et al., Expert Opin Biol Ther. 2013 April; 13(4): 10.1517/14712598.2013.758708) or skin inflammation in cutaneous T cell lymphoma (Guenova, et al., Clin Cancer Res. 2013.19: 3755-3763). See also Akdis, Nature Medicine 18, 736-749 (2012).

In some embodiments, collagen-targeted constructs as described herein that include an interferon-γ-binding domain can be used, e.g., to treat, reduce risk of, or prevent graft rejection, graft-versus-host disease, lupus, severe drug reactions and vitiligo (Pollard, et al., Discov Med. 2013. 16(87): 123-131 and Naisbitt, et al., Mol Pharmacol. 2003. 63(3): 732-741, Rashighi et al., Ann Transl Med. 2015. 3(21): 343).

In some embodiments, collagen-targeted constructs as described herein that include an IL-12-binding domain can be used, e.g., to treat graft rejection, graft-versus-host disease and severe drug reactions (Saito, et al., Eur J Immunol. 1996. 26(12): 3098-3106).

### Pharmaceutical Compositions and Methods of Administration

The methods described herein include the use of pharmaceutical compositions comprising a collagen VII-targeted construct as described herein as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into or administered with the compositions, e.g., anti-inflammatory agents such as corticosteroids, e.g., Hydrocortisone type steroids such as hydrocortisone, methylprednisolone, prednisolone, prednisone, and triamcinolone; Betamethasone type steroids such as beclometasone, betamethasone, dexamethasone, fluocortolone, halometasone, and mometasone and non-steroidal anti-inflammatory medications including inhibitors of phosphodiesterase 4 (PDE4), such as apremilast and roflumilast, and cyclophilin-binding drugs, such as tacrolimus and pimecrolimus.

Pharmaceutical compositions are typically formulated to be compatible with its intended route of administration. Examples of routes of administration include parenteral, e.g., intravenous, intradermal, intramuscular, subcutaneous, oral (e.g., inhalation), transdermal (topical), and transmucosal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, e.g., in embodiments for treatment, reduction of risk of, or prevention of an inflammatory condition of the lung, a therapeutic construct described herein can also be administered to a subject by way of the lung. Pulmonary drug delivery may be achieved by inhalation, and administration by inhalation herein may be oral and/or nasal. Examples of pharmaceutical devices for pulmonary delivery include metered dose inhalers, dry powder inhalers (DPIs), and nebulizers. For example, a therapeutic construct can be administered to the lungs of a subject by way of a dry powder inhaler. These inhalers are propellant-free devices that deliver dispersible and stable dry powder formulations to the lungs. Dry powder inhalers are well known in the art of medicine and include, without limitation: the TurboHaler® (AstraZeneca; London, England) the AIR® inhaler (Alkermes®; Cambridge, Mass.); Rotahaler® (GlaxoSmithKline; London, England); and Eclipse™ (Sanofi-Aventis; Paris, France). See also, e.g., PCT Publication Nos. WO 04/026380, WO 04/024156, and WO 01/78693. DPI devices have been used for pulmonary administration of polypeptides such as insulin and growth hormone. In some embodiments, a therapeutic construct described herein can be intrapulmonarily administered by way of a metered dose inhaler. These inhalers rely on a propellant to deliver a discrete dose of a compound to the lungs. Examples of compounds administered by metered dose inhalers include, e.g., Astovent® (Boehringer-Ingelheim; Ridgefield, Conn.) and Flovent® (GlaxoSmithKline). See also, e.g., U.S. Pat. Nos. 6,170,717; 5,447,150; and 6,095,141.

In some embodiments, a therapeutic construct described herein can be administered to the lungs of a subject by way of a nebulizer. Nebulizers use compressed air to deliver a compound as a liquefied aerosol or mist. A nebulizer can be, e.g., a jet nebulizer (e.g., air or liquid-jet nebulizers) or an ultrasonic nebulizer. Additional devices and intrapulmonary administration methods are set forth in, e.g., U.S. Patent Application Publication Nos. 20050271660 and 20090110679, the disclosures of each of which are incorporated herein by reference in their entirety. Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Dosage

An “effective amount” is an amount sufficient to effect beneficial or desired results. For example, a therapeutic amount is one that achieves the desired therapeutic effect (i.e., suppression of inflammatory response). This amount can be the same or different from a prophylactically effective amount, which is an amount necessary to delay or reduce risk of or prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Suitable human doses of any of the therapeutic constructs described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

In some embodiments, the present methods can include administering doses of a collagen VII-targeted construct that includes a therapeutic cytokine-biding domain that are significantly lower than doses required when the cytokine-biding domain is used alone (e.g., as low as 1% or 10% of the standard dose for the cytokine-binding domain alone).

### Kits

In some embodiments, a therapeutic construct or nucleic acid encoding such a construct is provided in the form of a kit, e.g., for use in a method described herein. In some embodiments, the kit comprises a packaged combination of reagents in predetermined amounts with instructions for performing a procedure. In some embodiments, the procedure is a therapeutic procedure. In other embodiments, the procedure is a diagnostic assay. In still other embodiments, the procedure is a research assay.

In some embodiments, the kit further comprises a solvent for the reconstitution of the therapeutic construct or nucleic acid. In some embodiments, the therapeutic construct or nucleic acid is provided in the form of a pharmaceutical composition. In some embodiments, the pharmaceutical composition is a lyophilized pharmaceutical composition.

The kits may comprise, in a suitable container, a therapeutic construct and/or nucleic acid, one or more controls, and various buffers, reagents, enzymes and other standard ingredients well known in the art.

The container can include at least one vial, well, test tube, flask, bottle, syringe, or other container means, into which a therapeutic construct and/or nucleic acid may be placed, and in some instances, suitably aliquoted. Where an additional component is provided, the kit can contain additional containers into which this component may be placed. The kits can also include a means for containing a therapeutic construct and/or nucleic acid and any other reagent containers in close confinement for commercial sale. Such containers may include injection or blow-molded plastic containers into which the desired vials are retained. Containers and/or kits can include labeling with instructions for use and/or warnings.

### Examples

The invention is further described in the following examples, which do not limit the scope of the invention described in the claims.

Materials and Methods

The following materials and methods were used in the Examples below.

Human Samples:

All studies were performed in accordance with the Declaration of Helsinki. Blood from healthy individuals was obtained after leukapheresis. Neonatal foreskins were obtained from infants undergoing circumcision at the Brigham and Women's Hospital. All tissues were collected with previous approval from the Institutional Review Board of the Partners Human Research Committee (Partners Research Management).

Preparation of Collagen VII-Streptavidin and Etanercept-Biotin:

Etanercept (Enbrel, Immunex Corporation, Thousand Oaks, Calif.) was purified using the Antibody Purification Kit Protein G then conjugated to biotin using the Biotin (type B) Conjugation Kit (Abcam, Cambridge, Mass.). Anti-human collagen VII (rabbit polyclonal, catalog # mbs2524211, MyBioSource Inc., San Diego, Calif.) was conjugated to streptavidin using the Streptavidin Conjugation Kit (Abcam.)

Human Engrafted Mouse Model:

Human neonatal foreskins were grafted onto the backs of 6- to 8-week old nonobese diabetic/severe combined immunodeficient/IL-2 receptor γ chain^{null} mice (NSG, Jackson Laboratory, Bar Harbor, Me.). Ten days later, 7×10⁶ allogeneic CD25 depleted PBMCs were injected intravenously. Six and 10 days after PBMC injection 20 ug etanercept-biotin, a mixture of 20 ug etanercept-biotin mixed with 20 ug anti-collagen VII-streptavidin, or saline control was injected intraperitoneally. Mice were sacrificed and skin grafts harvested for analysis three days after the last injection.

Immunohistochemical Studies:

Hematoxylin and eosin (H&E) staining was performed on formalin-fixed, paraffin-embedded (FFPE) tissue sections (4 μm) by standard immunohistochemical techniques. Human CD3 was detected in FFPE tissue sections (4 μm) by standard immunohistochemical techniques with anti-human CD3 (rabbit polyclonal, Dako # A0452, Santa Clara, Calif.) at a 1:250 dilution and 3,3′ diaminobenzidine (DAB) substrate.

Quantitative Real-Time PCR:

RNA was isolated from frozen, optimal cutting temperature (OCT) embedded skin samples. 30 cryosections of 10 μm thickness were cut and RNA extraction was carried out using the RNeasy Lipid Tissue Mini Kit (Qiagen, Germantown, Md.) kit as per manufacturer's instructions. 500 ug of total RNA from each sample was reverse transcribed to Complementary DNA using the SuperScript VILO cDNA Synthesis Kit (Life Technologies, Carlsbad, Calif.) and quantitative real-time PCR was performed using the ABI StepONE Plus instrument and the Fast SYBR green master mix (Life Technologies). Expression of each transcript was determined relative to the reference transcript ((3-actin) and calculated as 2\hat{ }−(C_{t, transcript(X)}−C_{t, β-actin}), then further normalized to the CD3ε transcript to account for potential differences in T cell infiltration into the grafts. (Moutsopoulos et al, J Autoimmun. 2012 December; 39(4):294-303). The primers used to detect the transcripts were purchased from Integrated DNA Technologies (Coralville, Iowa) and were as follows: CD3ε (F-5′-CCAGGATACTGAGGGCATGT-3′ (SEQ ID NO:48); R-5′-GGGGCAAGATGGTAATGAAG-3′(SEQ ID NO:49)), IL-17A (F-5′-CCACGAAATCCAGGATGCCCAAAT-3′(SEQ ID NO:50); R-5′-ATTCCAAGGTGAGGTGGATCGGTT-3′(SEQ ID NO:51)), IL-22 (F-5′-CACCAGTTGCTCGAGTTAGAA-3′(SEQ ID NO:52); R-5′-AAGGTGCGGTTGGTGATATAG-3′(SEQ ID NO:53)), IL-23A (F-5′-CCACACTGGATATGGGGAAC-3′(SEQ ID NO:54); R-5′-AGAAGCTCTGCACACTGGC-3′(SEQ ID NO:55)), Interferon gamma (IFNγ; F-5′-TGACCAGAGCATCCAAAAGA-3′(SEQ ID NO:56); R-5′-CTCTTCGACCTCGAAACAGC-3′(SEQ ID NO:57)), and β-actin (F-5′-TCACCCACACTGTGCCCATCTACGA-3(SEQ ID NO:58)′; R-5′-CAGCGGAACCGCTCATTGCCAATGG-3′(SEQ ID NO:59)).

### Example 1. Collagen VII-Targeted Anti-TNFα Effectively Suppressed Skin Inflammation

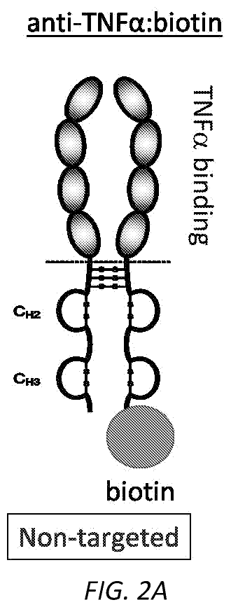

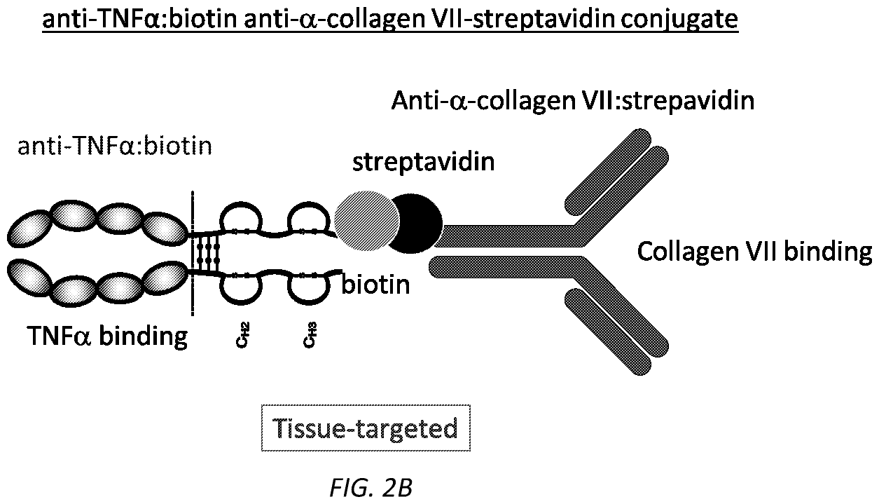

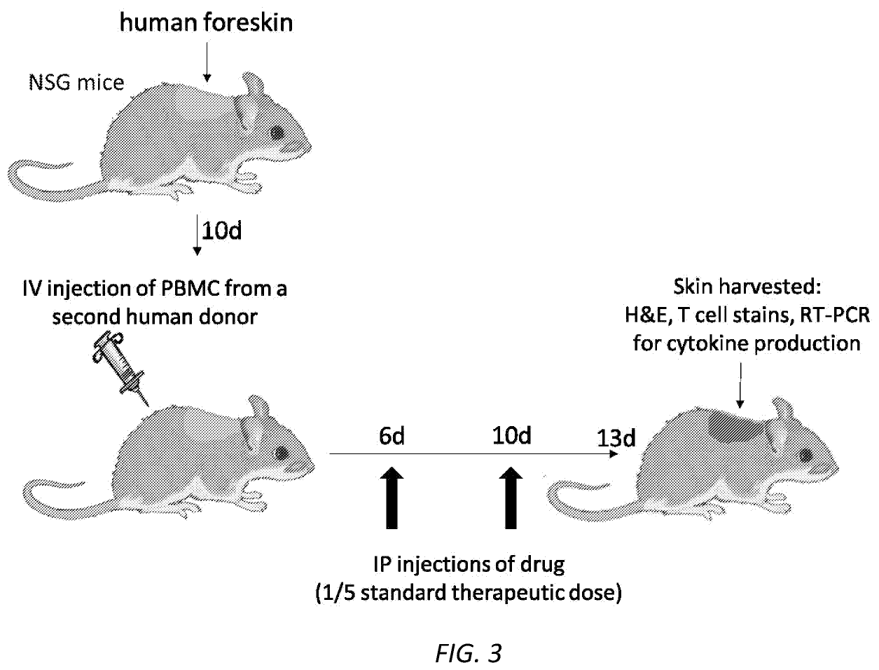

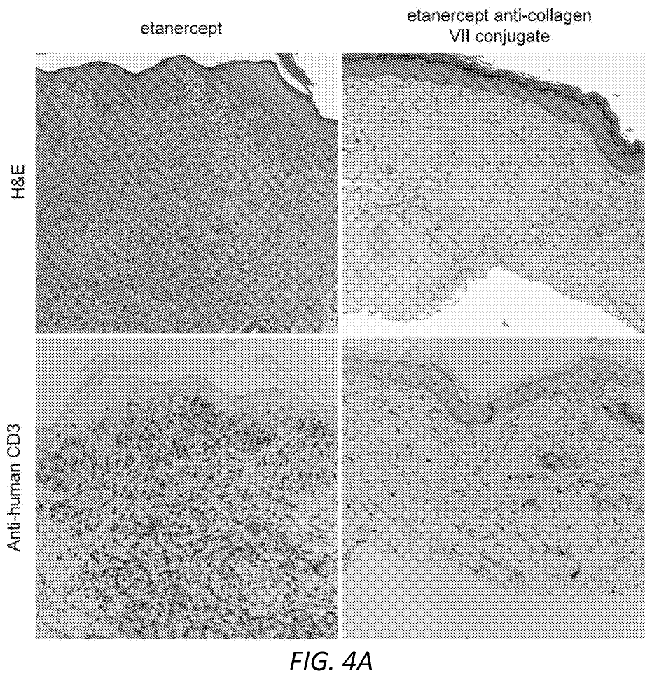

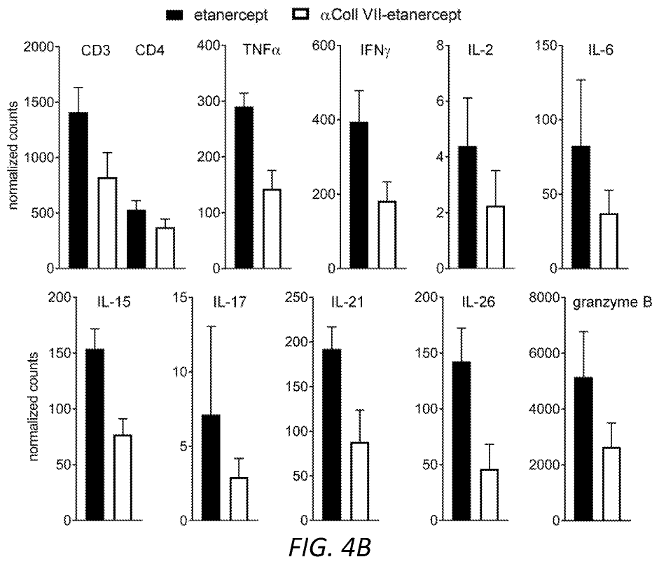

Pilot studies were carried out in NSG mice grafted with human neonatal foreskin, and injected i.v. with peripheral blood mononuclear cells (PBMC) from a second allogeneic human donor. These mice develop a graft versus host disease (GvHD)-like dermatitis within the grafted human skin, characterized by inflammatory T cell infiltrates, epidermal injury, dyskeratosis and dermal fibrosis. This model is a useful system in which to study human skin inflammation mediated by human T cells in an accessible animal model. To determine if a TNFα antagonist conjugated to an anti-collagen VII antibody (tissue targeted) could suppress skin inflammation at lower doses than are needed with the TNFα antagonist alone (non-targeted), a tissue targeted construct was generated by biotinylating the anti-TNFα biologic etanercept and allowing it to associate with a streptavidin-conjugated antibodies specific for human collagen VII (FIG. 2B). Mice were treated with intraperitoneal injections on days 6 and 10 after allogeneic PBMC infusion with subtherapeutic doses of either the anti-TNFα:biotin (20 μg, equivalent to about ⅕ the standard therapeutic dose) or the collagen VII-targeted conjugate (containing the same molar dose of anti-TNFα) as outlined in FIG. 3. 13 days after PBMC injection, human skin grafts were harvested from the mice and analyzed histologically (H&E stains, immunostaining for human T cells (CD3) cytokine production was in the skin was analyzed by real-time quantitative PCR. Mice injected with the tissue targeted conjugate had reduced inflammatory infiltrates, reduced epithelial damage, and decreased numbers of skin infiltrating CD3⁺ T cells (FIG. 4A). Mice injected with the tissue targeted conjugate also had reduced production of multiple inflammatory cytokines including TNFα, IFNgamma, IL-2, IL-6, IL-15, IL-17, IL-21, IL-26, as well as CD3, CD4, and granzyme B, in the skin (FIG. 4B). Taken together, these pilot studies demonstrated that a collagen VII targeted anti-TNFα biologic was able to effectively suppress skin inflammation at a lower dose than was required for the non-targeted biologic.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A collagen-targeted construct comprising:
(i) a collagen binding domain that binds specifically to human collagen VII; and
(ii) a cytokine binding domain that binds specifically to an inflammatory cytokine and inhibits binding of the cytokine to its receptor,
wherein the collagen binding domain and the cytokine binding domain are bound to each other.

2. The collagen-targeted construct of claim 1, wherein (i) and (ii) are each capable of binding to its cognate antigen at the same time.

3. The collagen-targeted construct of claim 1, wherein the cytokine binding domain binds specifically to an inflammatory cytokine selected from the group consisting of tumor necrosis factor alpha (TNFα), Interleukin 17A (IL-17A), IL-12, IL-23, IL-6, IL-4, and Interferon gamma (IFNγ).

4. The collagen-targeted construct of claim 1, wherein one or both of the collagen binding domain and the cytokine binding domain are antibodies or antigen-binding fragments thereof.

5. The collagen-targeted construct of claim 4, wherein the antibody or antigen-binding fragment thereof is an Fv fragment, a Fab fragment, a F(ab′)2 fragment, a Fab′ fragment, an scFv fragment, an scFv-Fc fragment, and/or a single-domain antibody or antigen binding fragment thereof.

6. The collagen-targeted construct of claim 4, which comprises an Fc domain that has reduced or no effector function.

7. The collagen-targeted construct of claim 4, wherein the antibody or antigen-binding fragment thereof is fully human, humanized, and/or chimeric.

8. The collagen-targeted construct of claim 1, which is a fusion protein.

9. The collagen-targeted construct of claim 1, wherein (i) and (ii) are joined via chemical conjugation.

10. A multispecific antibody comprising:
(a) first antigen-binding domain that specifically binds to human collagen VII and
(b) a second antigen-binding domain that (i) binds to an inflammatory cytokine and (ii) inhibits binding of the inflammatory cytokine to its cognate receptor.

11. The multispecific antibody of claim 10, which is a bispecific antibody.

12. The multispecific antibody of claim 10 or 11, wherein the second antigen-binding domain binds to an inflammatory cytokine selected from the group consisting of TNFα, IL-17A, IL-12, IL-23, IL-6, IL-4, and IFNγ.

13. A nucleic acid encoding the multispecific antibody of claim 10.

14. A method of reducing inflammation in the gut, lung, or skin of a subject, the method comprising administering a therapeutically effective amount of the multispecific antibody of claim 10, to a subject in need thereof.

15. A method of treating a subject who has a disorder associated with an inflammatory response in a tissue that expresses Collagen VII, the method comprising administering a therapeutically effective amount of the multispecific antibody of claim 10, to a subject in need thereof.

16. The method of claim 15, wherein the tissue is the lung, skin, or gut of the subject.

17. The method of claim 15, wherein the subject has rheumatoid arthritis, psoriasis, inflammatory bowel disease, asthma, atopic dermatitis, dermatomyositis, systemic or cutaneous lupus erythematosus, scleroderma, graft-versus-host disease, or organ transplant rejection after transplant of an organ that includes an epithelial layer.

18. A method of reducing immune cell infiltration at a site of inflammation in a subject, preferably wherein the site of inflammation is in the gut, skin, or lungs of the subject, the method comprising administering a therapeutically effective amount of the multispecific antibody of claim 10, to a subject in need thereof.

19. A method of inhibiting inflammatory cytokine activity at a site of inflammation in a subject, preferably wherein the site of inflammation is in the gut, skin, or lungs of the subject, the method comprising administering a therapeutically effective amount of the multispecific antibody of claim 10, to a subject in need thereof.

20. A method of treating skin inflammation in a subject, the method comprising administering a therapeutically effective amount of the multispecific antibody of claim 10, to the skin of a subject in need thereof.

21. The method of claim 20, wherein the subject has psoriasis, atopic dermatitis, dermatomyositis, systemic or cutaneous lupus erythematosus, scleroderma, graft-versus-host disease, or organ transplant rejection after a skin transplant.

22. A method of treating lung inflammation in a subject, the method comprising administering a therapeutically effective amount of the multispecific antibody of claim 10, to the lungs of a subject in need thereof.

23. The method of claim 22, wherein the subject has asthma, graft-versus-host disease, or organ transplant rejection after transplant of a lung.

24. A method of treating gut inflammation in a subject, the method comprising administering a therapeutically effective amount of the multispecific antibody of claim 10, to the gut of a subject in need thereof.

25. The method of claim 24, wherein the subject has inflammatory bowel disease.

26.-37. (canceled)


SEQUENCE LISTING (59 sequences)

SEQ ID NO: 1 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 2 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 4 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 5 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 6 - protein, 136 aa, Homo sapiens
MCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 7 - protein, 153 aa, Homo sapiens [sequence omitted]

SEQ ID NO: 8 - protein, 137 aa, Homo sapiens
MGLTSQLLPPLFFLLACAGNFVHGHKCDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 9 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 11 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 12 - protein, 467 aa, Artificial Sequence
  Description of Artificial Sequence Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 226 aa, Artificial Sequence
  Description of Artificial Sequence Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 14 - protein, 214 aa, Artificial Sequence
  Description of Artificial Sequence Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 219 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLDMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKI

SEQ ID NO: 16 - protein, 213 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 17 - protein, 126 aa, Artificial Sequence
  Description of Artificial Sequence Synthetic polypeptide
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 18 - protein, 108 aa, Artificial Sequence
  Description of Artificial Sequence Synthetic polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 19 - protein, 118 aa, Artificial Sequence
  Description of Artificial Sequence Synthetic polypeptide
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSS

SEQ ID NO: 20 - protein, 107 aa, Artificial Sequence
  Description of Artificial Sequence Synthetic polypeptide
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIK

SEQ ID NO: 21 - protein, 445 aa, Artificial Sequence
  Description of Artificial Sequence Synthetic polypeptide
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 219 aa, Artificial Sequence
  Description of Artificial Sequence Synthetic polypeptide
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 127 aa, Artificial Sequence [sequence omitted]

SEQ ID NO: 24 - protein, 109 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 25 - protein, 449 aa, Artificial Sequence
  Description of Artificial Sequence Synthetic polypeptide
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 446 aa, Artificial Sequence
  Description of Artificial Sequence Synthetic polypeptide
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 217 aa, Artificial Sequence
  Description of Artificial Sequence Synthetic polypeptide
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 449 aa, Artificial Sequence
  Description of Artificial Sequence Synthetic polypeptide
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 213 aa, Artificial Sequence
  Description of Artificial Sequence Synthetic polypeptide
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 99 aa, Artificial Sequence
  Description of Artificial Sequence Synthetic polypeptide
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNA

SEQ ID NO: 32 - protein, 109 aa, Artificial Sequence
  Description of Artificial Sequence Synthetic polypeptide
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNL

SEQ ID NO: 33 - protein, 220 aa, Artificial Sequence
  Description of Artificial Sequence Synthetic polypeptide
EVQLVESGGGLVQPGGSLRLSCAASGFNFNDYFMNWVRQAPGKGLEWVAQMRNKNYQYGTYYAESLEGRFTISRDDSKNSLYLQMNSLKTEDTAVYYCARESYYGFTSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVES

SEQ ID NO: 34 - protein, 214 aa, Artificial Sequence
  Description of Artificial Sequence Synthetic polypeptide
DIQMTQSPSSLSASVGDRVTITCQASQDIGISLSWYQQKPGKAPKLLIYNANNLADGVPSRFSGSGSGTDFTLTISSLQPEDFATYYCLQHNSAPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 451 aa, Artificial Sequence
  Description of Artificial Sequence Synthetic polypeptide
QVTLRESGPALVKPTQTLTLTCTFSGFSLSTSGMGVSWIRQPPGKGLEWLAHIYWDDDKRYNPSLKSRLTISKDTSRNQVVLTMTNMDPVDTATYYCARRETVFYWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKRVVSVLPREEQYNSTYTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 218 aa, Artificial Sequence
  Description of Artificial Sequence Synthetic polypeptide
DIVLTDRVTIQSPSSLSASVGTCKASQSVDYDGDSYMNWYQQKPGKAPKLLIYAASNLESGIPSRFSGSGSGTDFTFTISSLQPEEDPPTDFIATYGQGTYCQQSNKVEIKRTVAAPSVFIFPPSDEQLKSGTASVVCLLNNFYPREAKVQWKVDNALQSGNSQEYSLSSHKVYASVTEQDSKDSTTLTLCEVTSKADYEKHQGLSSPVTKSFNRGEC

SEQ ID NO: 37 - protein, 445 aa, Artificial Sequence
  Description of Artificial Sequence Synthetic polypeptide
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 217 aa, Artificial Sequence
  Description of Artificial Sequence Synthetic polypeptide
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 5 aa, Artificial Sequence
  Description of Artificial Sequence Synthetic peptide
GGGGS

SEQ ID NO: 40 - protein, 26 aa, Artificial Sequence
  Description of Artificial Sequence Synthetic peptide
NSTYRVVSVLTVLHQDWLNGKEYKCK

SEQ ID NO: 41 - protein, 25 aa, Artificial Sequence
  Description of Artificial Sequence Synthetic peptide
FQNALLVRYTKKVPQVSTPTLVEVS

SEQ ID NO: 42 - protein, 5 aa, Artificial Sequence
  Description of Artificial Sequence Synthetic peptide
GGGGS

SEQ ID NO: 43 - protein, 10 aa, Artificial Sequence
  Description of Artificial Sequence Synthetic peptide
GGGGSGGGGS

SEQ ID NO: 44 - protein, 15 aa, Artificial Sequence
  Description of Artificial Sequence Synthetic peptide
GGGGSGGGGSGGGGS

SEQ ID NO: 45 - protein, 20 aa, Artificial Sequence
  Description of Artificial Sequence Synthetic peptide
GGGGSGGGGSGGGGSGGGGS

SEQ ID NO: 46 - protein, 25 aa, Artificial Sequence
  Description of Artificial Sequence Synthetic peptide
GGGGSGGGGSGGGGSGGGGSGGGGS

SEQ ID NO: 47 - protein, 30 aa, Artificial Sequence
  Description of Artificial Sequence Synthetic polypeptide
GGGGSGGGGSGGGGSGGGGSGGGGSGGGGS

SEQ ID NO: 48 - DNA, 20 bp, Artificial Sequence
  Description of Artificial Sequence Synthetic primer
ccaggatactgagggcatgt

SEQ ID NO: 49 - DNA, 20 bp, Artificial Sequence
  Description of Artificial Sequence Synthetic primer
ggggcaagatggtaatgaag

SEQ ID NO: 50 - DNA, 24 bp, Artificial Sequence
  Description of Artificial Sequence Synthetic primer
ccacgaaatccaggatgcccaaat

SEQ ID NO: 51 - DNA, 24 bp, Artificial Sequence
  Description of Artificial Sequence Synthetic primer
attccaaggtgaggtggatcggtt

SEQ ID NO: 52 - DNA, 21 bp, Artificial Sequence
  Description of Artificial Sequence Synthetic primer
caccagttgctcgagttagaa

SEQ ID NO: 53 - DNA, 21 bp, Artificial Sequence
  Description of Artificial Sequence Synthetic primer
aaggtgcggttggtgatatag

SEQ ID NO: 54 - DNA, 20 bp, Artificial Sequence
  Description of Artificial Sequence Synthetic primer
ccacactggatatggggaac

SEQ ID NO: 55 - DNA, 19 bp, Artificial Sequence
  Description of Artificial Sequence Synthetic primer
agaagctctgcacactggc

SEQ ID NO: 56 - DNA, 20 bp, Artificial Sequence
  Description of Artificial Sequence Synthetic primer
tgaccagagcatccaaaaga

SEQ ID NO: 57 - DNA, 20 bp, Artificial Sequence
  Description of Artificial Sequence Synthetic primer
ctcttcgacctcgaaacagc

SEQ ID NO: 58 - DNA, 25 bp, Artificial Sequence
  Description of Artificial Sequence Synthetic primer
tcacccacactgtgcccatctacga

SEQ ID NO: 59 - DNA, 25 bp, Artificial Sequence
  Description of Artificial Sequence Synthetic primer
cagcggaaccgctcattgccaatgg